(19) United States
(12) Patent Application Publication
(10) Pub. No.: US 2026/0048043 A1
(43) Pub. Date: Feb. 19, 2026

(54) HIGH LOADING ORAL FILM FORMULATION WITH IMPROVED BIOAVAILABILITY

(71) Applicant: INTELGENX CORP., ST-LAURENT (CA)

(72) Inventors: Billal TIR, Montréal (CA); Nadine PAIEMENT, St-Laurent (CA)

(21) Appl. No.: 19/102,093

(22) PCT Filed: Aug. 10, 2023
(86) PCT No.: PCT/CA2023/051069

§ 371 Date: Feb. 7, 2025

Related U.S. Application Data
(60) Provisional application No. 63/396,956, filed on Aug. 10, 2022.

(51) Int. Cl. A61K 31/439; A61K 9/00; A61K 47/10; A61K 47/24; A61K 47/26; A61K 47/46

(52) U.S. Cl. CPC A61K 31/439; A61K 9/006; A61K 47/10; A61K 47/24; A61K 47/26; A61K 47/46

(57) ABSTRACT

Oral film formulations are disclosed in the present application. The oral film formulations comprise: i. suspended Maropitant or a salt thereof: ii. an aqueous phase consisting of water and at least one surfactant: iii. at least one viscosity increasing agent/suspending agent and iv. at least one solubilizer.

### CROSS-REFERENCE TO RELATED APPLICATIONS

The present application claims priority to U.S. provisional application No. 63/396,956 filed on Aug. 10, 2022. This document is hereby incorporated by reference in its entirety.

### FIELD OF THE DISCLOSURE

This disclosure relates to oral film dosage formulations and processes for preparing oral film dosage forms, and more particularly to the preparation of oral film dosage forms that are suitable for high active content films for both human and animal applications that exhibit improved bioavailability.

### BACKGROUND OF THE DISCLOSURE

It is often desirable to administer a pharmaceutical ingredient using an oral film dosage form. Oral film dosage forms have several advantages when compared with tablets and capsules. Many people have difficulty swallowing tablets and capsules, and risk choking while attempting to swallow solid oral dosage forms, but can self-administer a film dosage form without difficulty. Similarly, administering drugs through various dosage forms to animals, such as companion animals, often presents unique challenges, especially where dosing precision is desired. Animals may reject portions of tablets some of which are required to be split in several pieces amplifying dosing imprecision.

While administration of a drug in an oral film dosage form can be desirable, designing an oral dosage form that provides a desirable absorption profile remains a challenge. Oral film formulations have the potential to enhance the rate of absorption of a drug or active pharmaceutical ingredient (API). Despite the desired advantages of oral film formulations, suitable oral film formulations have so far had limited applicability in part due to the limited API loading capability of oral films. The low API loading of oral films is derived from several factors inherent to oral films such as the size limitation dictated by pharmacokinetics but also by the size of a person's or animal's mouth. Oral film mechanical properties also limit the API content to provide suitable flexibility parameters for the oral film packaging process. These unfortunate limitations of oral films have left the potential of oral film formulation relatively unexploited which is apparent by the limited number of approved drugs having oral film dosage forms.

The use of maropitant for treatment of emesis and related afflictions is not new in the art. WO2005/082416 describes the development of a pharmaceutical formulation of maropitant citrate, sulfobutyl ether β-cyclodextrin and meta-cresol. The dosage method in this case is injection through the skin which can lead to pain and adverse reactions in animal subjects. EP3173071A1 relates to a pharmaceutical compositions comprising maropitant, a method for preparing the composition, the use of the composition as a medicament, and the use of the composition in the treatment of emesis in mammals, in particular cats and dogs. The dosage method in this case improves the administration method of injection but again does not contemplate other dosage administration alternatives such as oral films. CN110577522A relates to a new crystal form of maropitant citrate and a preparation method thereof, with no mention of using films as an administration method.

Some oral administration methods of treatments compositions for emesis and related afflictions in animals exist in the art, such as CA2965524, where the oral form is a soft, chewable composition that does not bypass hepatic circulation to the extent that oral films do, and generally employs different composition techniques and biological systems.

Oral films for veterinary use also exist in the art such as US20040115253, US 20060121096, US20140286876, US20160166511. There exists a need for effective high loading animal health films with improved bioavailability.

Oral film technology can provide an alternative injectable and tablet dosage forms and to accommodate subjects or pet owners by providing more easily administrable medicines without the need for them to go to the clinic, experience pain or to embed the dosage form in food. There also exists a need for increased bioavailability of high concentration of active agents in treatment, and increased compliance to treatment by improving the administration and acceptability of the product.

These and other inefficiencies and opportunities for improvement are addressed and/or at least partially overcome by the systems, assemblies and methods of the present disclosure.

### SUMMARY OF THE DISCLOSURE

In general, an oral film dosage form includes solubilized API in solubilizer, permeation co-enhancer, suspended API, a surfactant system, a suspending agent/viscosity increasing agent, plasticizer, flavor, taste masking agent and polymeric film matrix.

According to some aspects of the disclosure, the oral film dosage form comprises at least one API in a dual system: API in solubilizer(s) and suspended API in polymeric mucoadhesive film matrix.

In certain aspects of this disclosure, the disclosed formulations and excipients are specifically adapted for use in humans.

In certain aspects of this disclosure, the disclosed formulations and excipients are specifically adapted for use in animals.

These and other features, advantages and objects of the various embodiments will be better understood with reference to the following specification and claims.

### BRIEF DESCRIPTION OF THE FIGURES

FIG. 1 demonstrates the permeation test of three Maropitant oral films through pig buccal mucosa. The permeation test uses Franz Cell equipment where the donor media is purified water and the receiver media is 30% hydroxypropyl-beta-cyclodextrin in purified water. The results of maropitant concentration in the receiver (ug/cm2) demonstrate the superiority of the oral film where maropitant is solubilized in acidic conditions (lot 164-24-2) followed by oral films where maropitant is partially solubilized in oleic acid and benzyl alcohol (lot 164-28-3B), and finally the last profile of oral films where the majority of maropitant is suspended (lot 164-44). It is evident that the solubility of the API improves its permeability.

FIG. 2 demonstrates the said permeation test as in FIG. 1 where comparing the effect of permeation co-enhancers (glycerol in lot 164-28-3A versus PEG 300 on lot 164-28-3B) on the permeation of maropitant oral films containing two solubilizers oleic acid and benzyl alcohol.

FIG. 3 demonstrates the said permeation test as in FIG. 1, demonstrating the improvement of the permeability when using the solubilizer oleic acid in lot 164-5E during the first five hours compared to benzyl acetate in lot 164-25-1 and benzyl alcohol in lot 164-15-2.

FIG. 4 demonstrates the said permeation test as in FIG. 1. FIG. 4 shows the improvement of maropitant permeability in lot 164-28-3A when increasing the amount of surfactant tween 20 and labrafil with the addition of water soluble solubilizer (benzyl alcohol) to maropitant oral films already employing oleic acid as solubilizer (lot 164-28-6A.) FIG. 4 shows the improvement of maropitant permeability and the reduction of lag time in lot 164-28-3B when switching the permeation co-enhancer glycerol (lot 164-28-3A) to PEG 300. FIG. 4 demonstrates the improvement of maropitant permeability and the reduction of lag time in lot 164-28-6B when switching the permeation co-enhancer glycerol (lot 164-28-6A) to the propylene glycol. The optimization of lot 164-28-6A by adding more tween and labrafil with the addition of benzyl alcohol will generate the lot 164-28-3A where the latter gives a similar permeation profile as in lot 164-28-6B that has only the permeation co-enhancer that is switched from glycerol (lot 164-28-6A) to propylene glycol (lot 164-28-6B.) Therefore the said improvement of lot 164-28-3A to lot 164-28-3B generates a better permeation profile than the lot 164-28-6B.

FIG. 5 demonstrates the said permeation test as in FIG. 1. The FIG. 5 shows the improvement of maropitant permeability in lot 164-5E when increasing the amount of the solubilizer (oleic acid) compared to lot 164-28-6A.

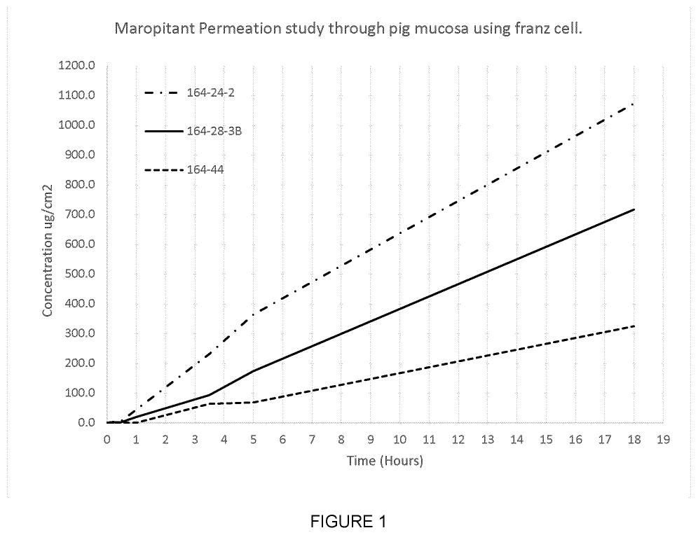

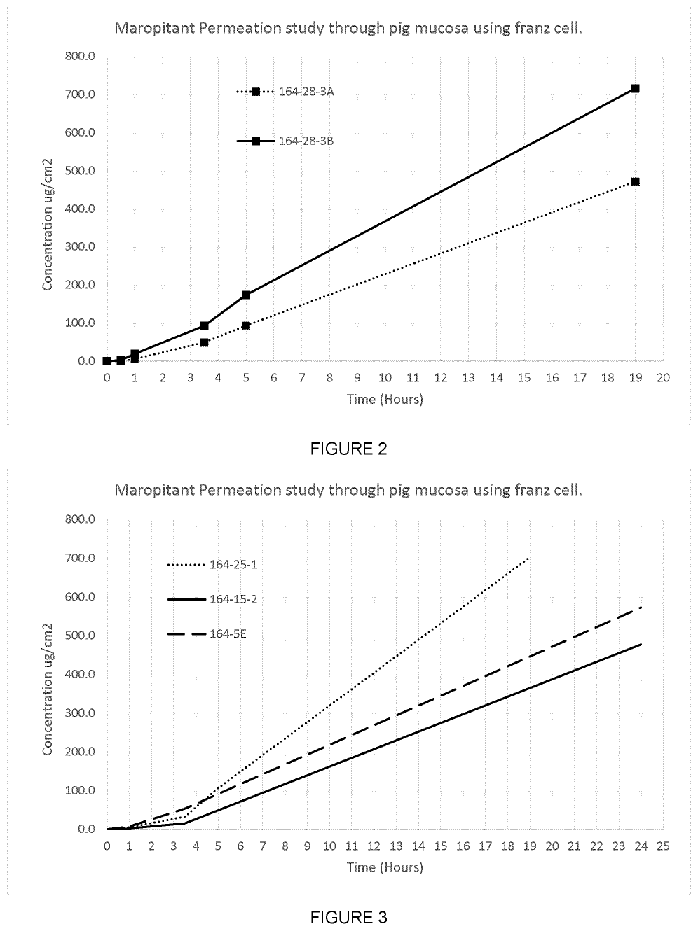

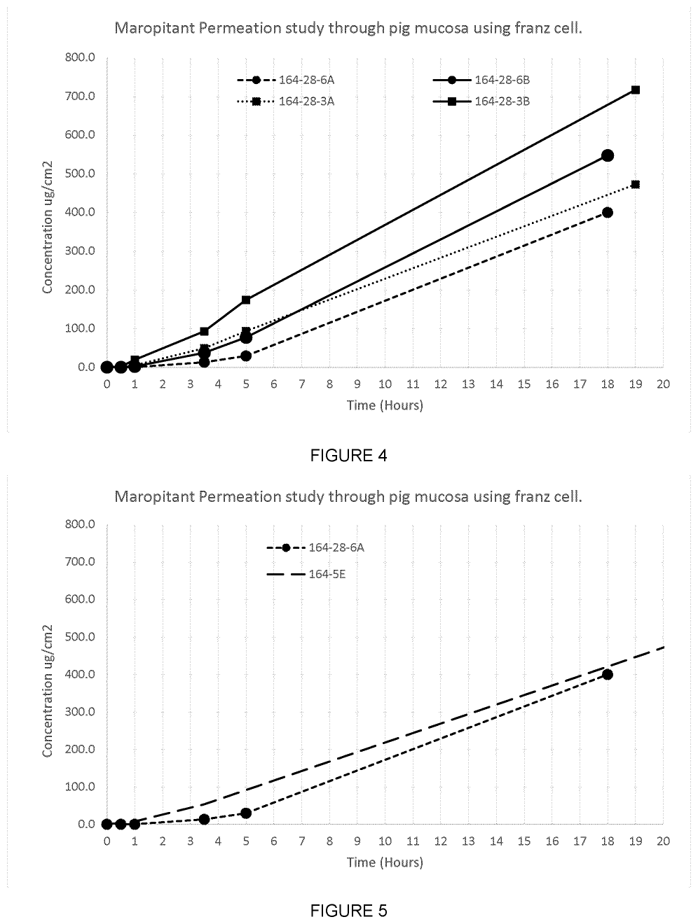

### DETAILED DESCRIPTION

Unless defined otherwise, all technical and scientific terms used herein have the same meaning as commonly understood by one of ordinary skill in the art.

It is to be understood that this disclosure is not limited to particular embodiments described, and as such may, of course, vary. It is also to be understood that the terminology used herein is for the purpose of describing particular embodiments only, and is not intended to be limiting.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The term “about” as used herein can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

Film systems embody a field of technology that has major advantages in areas of administering various active agents to an individual or subject in need thereof. The present disclosure relates to oral films, also referred to in the art as films and film strips, sheets, discs, wafers, and the like, in any shape, including rectangular, square, or other desired shape, and methods for forming film products that include at least one active agent. Specifically, the disclosure provides for a film and a method of forming a film. The terms “oral dissolving film,” “oral dissolvable film”, “oral disintegrating film”, OSF, “oral soluble film”, “OCF”, “oral chewable film”, “ODF”, “oral dissolvable film”, “OTF,” “oral thin film”, “oral strip” and the like refer to a product used to administer a predetermined amount of active ingredient(s) via oral route such as oral transmucosal absorption, sublingual delivery or buccal delivery and will be referred to throughout as “oral film(s)”.

The term “active agent(s)” or “API” refers mainly to active pharmaceutical ingredients, drugs, pharmaceuticals, but may also refer generally to any agent(s) that is intended for incorporation into a finished drug product and is intended to furnish pharmacological activity or other direct effect in the diagnosis, cure, mitigation, treatment, or prevention of disease, or to affect the structure or any function of the body subject.

A pharmaceutical composition can include one or more pharmaceutically active components. The pharmaceutically active component can be a single pharmaceutical component or a combination of pharmaceutical components. The pharmaceutically active component can be an anti-inflammatory analgesic agent, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, an antirheumatic, an antihypertensive, a bronchodilator, an anticholinergic, an anti-anxiety drug, an antiemetic compound, a hormone, a peptide, a protein or a vaccine. The pharmaceutically active component can be the compound, pharmaceutically acceptable salt of a drug, a prodrug, a derivative, a drug complex or analog of a drug. The term “prodrug” refers to a biologically inactive compound that can be metabolized in the body to produce a biologically active drug.

In some embodiments, more than one pharmaceutically active component may be included in the film. The pharmaceutically active components can be ace-inhibitors, anti-anginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, amphetamines, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, psychedelics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, diagnostic agents, imaging agents, dyes, or tracers, and combinations thereof.

The film product of the present disclosure includes an active component selected from pharmaceutical agents, bioactive agents, cosmetic agents, and combinations thereof. The active component may be present in any desired amount effective for the intended treatment. It is particularly desirable and an advantage of the present disclosure that the active component can be included at high loading. Bioactive agents for oral films are substances with specific biological effects when administered orally, often used to achieve therapeutic outcomes in pets or humans. These agents encompass pharmaceutical drugs, vitamins, minerals, nutraceuticals, enzymes, probiotics, hormones, and more. Incorporated into oral film formulations, they target conditions like pain, inflammation, infection, or support general wellness. Considerations include stability, bioavailability, and controlled release to optimize the agents' effects while providing a convenient and effective administration method.

A variety of additives that can be integrated into the films may provide a variety of different functions. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added along with the active ingredient(s)”.

The term “matrix” or “film matrix” refers to the polymer component or mixture of polymers, which creates the “film-forming matrix” supporting the API within the oral film dosage form.

The term “amorphous” refers to the non-crystalline form of the solid, a state that lacks the regular crystalline organization of atoms. The amorphous content (amorphicity) of a solid can be accurately and precisely assessed using a number of well-established methodologies, including isothermal calorimetry, Powder X-ray Diffraction (PXRD), Differential Scanning calorimetry (DSC), Continuous Relative Humidity Perfusion, Microcalorimetry (cRHp), and Dynamic Vapor Sorption (DVS).

The term “stable” refers to a product which exhibits no changes in the dissolution profile or remains within the established specifications and recovery when the product is exposed to normal stability conditions (e.g., 25° C./60% RH and 40° C./75% RH) for an extended period of time while also demonstrating no chemical degradation. The term “stable” can also refer also to mechanical stability, such as in the case where the product is recrystallizing, there will be a change in flexibility and other mechanical properties. The term “stable” can also refer also to chemical stability and refers to a product which exhibits changes in the assay and impurity profile that remains within the established specifications when the product is exposed to normal stability conditions (e.g., 24 months at 25° C./60% RH and 6 months at 40° C./75% RH) demonstrating chemical degradation within established specifications.

The term “non-solubilized” means that the crystalline, solid amorphous powder or partially amorphous active agent(s) is (are) suspended (insoluble) to form a homogeneous solution with another substance, the solvent. The stability of an API can be increased in a finished film product by using the API as a partially/non-solubilized dispersion. A solubilized API, particularly a crystalline API, may re-crystallize over time, during time and storage conditions, which may adversely affect the overall bioavailability of the product. The choice of a partially/non-solubilized API can also be used to control the dissolution behavior and release of API from a film dosage for systemic uptake within a patient. Uptake and absorption of API is governed by the drug solubility and availability; thus controlling its crystallinity and particle size allows us to influence the bioavailability of the API within the human or animal body.

The mean particle size diameter (D50) equal or lower than 250 μm, refers to the size distribution of the solid particle uniformly distributed in the matrix film. The size can be small enough to avoid any rough texture or unpleasant mouth feel experience when orally ingested.

The term “suspended” (and variations thereof) refers to a dispersion of solid material (e.g., particles or powder) in a bulk liquid medium, in which the solid material is not completely dissolved on a molecular level, and will eventually settle out of the liquid in the absence of agitation or sufficient viscosity of the blend. In a suspension, the suspended material is not completely dissolved in the liquid.

The term “polymer” refers to a long molecule chain made of many repeating units. A “film forming polymer” refers to a polymer used in the forming and manufacture of a film matrix.

The term “water soluble ingredient” refers to an ingredient able to be dissolved in water.

The term “non-water soluble ingredient” refers to ingredients generally unable to be dissolved in water.

The terms “suspending agent” (also referred to as a “viscosity increasing agent”) refers to water soluble ingredients or non-water soluble ingredients or a combination thereof employed to prevent adjacent suspended particles from coming close enough to join each other by sufficiently increasing the viscosity of the drug vehicle, and enabling by steric stabilization the suspension (suspended entities) to be stably maintained. Certain suspending agent/viscosity increasing agent additionally interact with biological mucosa to create a strengthened oral film mucoadhesion. Examples include polysaccharides in the form of one or a mix of the following and their derivatives such as Hydroxypropylmethylcellulose (HPMC) where the polymer structure combines both hydrophobic (methoxy group) and hydrophilic substitutions (hydroxypropoxy group) where the 2% aqueous viscosity is between about 1298 to about 15000 millipascal second (mPas) (2%, 20C), Hydroxypropyl Cellulose (HPC) where the 2% aqueous viscosity is above about 150 mPas (2%, 25 C), hydroxyethyl cellulose (HEC), gums such as water soluble carboxymethyl cellulose (CMC), gellan, propylene glycol alginate, water soluble alginate salt, acacia, pectin, xanthan, guar gum, carrageenan, and water insoluble alginates derivatives, water insoluble CMC derivatives, colloidal silicon dioxide, Agar, Locust bean and tragacanth. The definition also comprises Polyvinylpyrrolidone of Molecular Weight (MW) of 1 000 000 MW and above (K-value of 85 and above) with aqueous viscosity of 300 mPAs (10%, 20 C) and above and higher molecular weight polyethylene oxide (PEO) (MW above 600 000). The following are excluded from the definition of the terms “suspending agent/viscosity increasing agent”: one or a mix of HPMC where the polymer structure do not combines both hydrophobic and hydrophilic substitutions, and or having aqueous viscosity below 1298 mPas or above 15000 mPas (2%, 20 C), Methyl cellulose (MC), Microcrystalline cellulose (MCC), powdered cellulose, Sodium Starch Glycolate, starch, Polyvinylpyrrolidone of MW below 1.000.000 MW and K-value below 85 and with aqueous viscosity of less than 300 mPAs (10%, 20 C), polyvinylpyrrolidone-vinyl acetate copolymer, polyplasdone crospovidone, HPC where the 2% aqueous viscosity is below 150 mPas (2%, 25 C), water insoluble bentonite.

The term “surfactant” or “surfactant system” refers to amphiphilic structure, with polar hydrophilic head (ionic or no-ionic) and non-polar hydrophobic tail. This balance between hydrophilic and lipophilic parts of amphiphilic molecules is expressed as the hydrophilic-lipophilic balance (HLB). Surfactants are employed to dissipate the free surface energy of particles by reducing the interfacial tension and contact angle between the solid and the suspending vehicle and comprise PEG 300 oleic glycerides (Labrafil® M-1944CS), PEG 300 linoleic glycerides (Labrafil® M-2125CS); Hydroxylated lecithin; Caprylocaproyl polyoxyl-8 glycerides; Polyoxyethylene (4) sorbitan monostearate, Polyoxyethylene 20 sorbitan tristearate, Polyoxyethylene (5) sorbitan monooleate, Polyoxyethylene 20 sorbitan trioleate; Sorbitan Esters (Sorbitan Fatty Acid Esters) such as: Sorbitan monolaurate, Polyoxyethylene Sorbitan Fatty Acid Esters such as: Polyoxyethylene 20 sorbitan monolaurate, Polyoxyethylene (4) sorbitan monolaurate, Polyoxyethylene 20 sorbitan monopalmitate, Polyoxyethylene 20 sorbitan monostearate, Polyoxyethylene 20 sorbitan monooleate, Polyoxyethylene 20 sorbitan monoisostearate, Polyethylene glycol monostearate (Gelucire 48/16), poloxamer having MW from 2200 up to 14.600, viscosity from 700 to 3100 mPAs (as a melt at 77 C) but exclude surfactants of an HLB below 7 such as Propylene glycol monocaprylate type I, Propylene glycol monocaprylate type II, Propylene glycol monolaurate, Sorbitan monoisostearate, Sorbitan monooleate, Sorbitan monopalmitate, Sorbitan monostearate, Sorbitan sesquioleate, Sorbitan trioleate, Sorbitan tristearate and glyceryl monoleate.

The term “co-surfactant” refers to a chemical substance that is used in addition to a surfactant to improve its performance especially: therefore, a second surfactant that is used in conjunction with a primary surfactant. Co-surfactants are usually alcohols, amines ranging from C4 to C10 or a molecule with polar hydrophilic head (ionic or no-ionic) and non-polar hydrophobic tail. The co-surfactant helps in the formation and stabilization of micelles/microemulsions.

The term “solubility enhancer” refers to excipients used to solubilize low solubility drugs via non-covalent interactions, and permit dissolution and bioavailability enhancement of the said drug. Non-covalent interactions include van der Waals forces, hydrogen bonding, dipole-dipole and ion-dipole interactions, and in certain cases favorable electromagnetic interactions. In this disclosure, the solubility enhancer involves one or combination of two types of excipients: type (I) of amphiphilic structure having both hydrophobic and hydrophilic constituents and type (II) of non-amphiphilic structure having either a majority of hydrophilic constituents or a majority of hydrophobic constituents. Solubility enhancers are divided into two categories, the first being the amphiphilic solubility enhancers type (I): A) Cellulosic derivative such as but not limited to HPMC having aqueous viscosity of Not More Than (NMT) 500 mPas (2%, 25 C) and HPC of low Molecular Weight (MW) Hydroxypropyl Cellulose (HPC) (up to 95.000) having aqueous viscosity of Not More Than (NMT) 150 mPas (5%, 25 C), and B) Surfactant(s) of HLB from 3 to 7 (category I) or of an HLB equal to 7 and above or a combination of the two categories: examples include but are not limited to sodium lauryl sulfate, copolymers of poly(ethylene oxide) (PEO) and poly(propylene oxide) (PPO) i.e poloxamer having MW up to 14.600 and viscosity up to 3100 mPas (77 C); PEG 300 oleic glycerides, PEG 300 linoleic glycerides; Sorbitan Esters (Sorbitan Fatty Acid Esters) such as: Sorbitan monoisostearate, Sorbitan monolaurate, Sorbitan monooleate, Sorbitan monopalmitate, Sorbitan monostearate, Sorbitan sesquioleate; Polyoxyethylene Sorbitan Fatty Acid Esters such as: Polyoxyethylene 20 sorbitan monolaurate, Polyoxyethylene (4) sorbitan monolaurate, Polyoxyethylene 20 sorbitan monopalmitate, Polyoxyethylene 20 sorbitan monostearate, Polyoxyethylene (4) sorbitan monostearate, Polyoxyethylene 20 sorbitan tristearate, Polyoxyethylene 20 sorbitan monooleate, Polyoxyethylene (5) sorbitan monooleate, Polyoxyethylene 20 sorbitan trioleate, Polyoxyethylene 20 sorbitan monoisostearate Propylene glycol monocaprylate type I and type II, Caprylocaproyl polyoxyl-8 glycerides, C) Polyvinylpyrrolidone of up to 1.500.000 MW, having aqueous viscosity of NMT 700 mPAs (10%, 20 C) D) polyethylene oxide (PEO), of up to 300.000 MW, having aqueous viscosity of NMT 1200 mPAs (5%, 25 C) and E) Cyclodextrines and their derivatives. The second category of Non-Amphiphilic solubility enhancers type (II): A) with majority of hydrophilic constituents: examples are not limited to Glycerol, Propylene glycol, and PEGs. Said PEGs of up to 6600 MW and of viscosity of Not More Than (NMT) 390 mPas (at 98.98 C+/−0.3 C) B) With majority of hydrophobic constituents examples are not limited to oily surfactant of lower Hydrophilic Lipophilic Balance (HLB) below 3 and oily solubility enhancer: Medium chain triglycerides (MCT) and Glycerol monolinoleate (Maisin CC TM), soybean oil, Olive oil, Sorbitan trioleate, Sorbitan tristearate.

The term “solubilizer” also includes all excipients acting as “solubility enhancer” and solubilizing a higher ratio of API compared to the present “solubility enhancer”. In this disclosure, the solubilizers involve one or the combination of: 1) non-volatile solubilizer(s) of more lipophilic character of log p less than 8 such as surfactants(s) of an HLB up to 7 (category I), fatty acids, oils or other lipid base excipient, of which non-limiting examples are alpha-linolenic acid, alpha-Linoleic acid, oleic acid, olive oil, sesame oil, palm oil. 2) Non-volatile solubilizer(s) of hydrophilic character of log p less than 5 such as but not limited to surfactant(s) of an HLB equal to 7 and above (category II), benzene derivatives as benzaldehyde, benzyl acetate, benzyl benzoate, pH modulators such as but not limited to hydrochloric acid, citric acid, sodium hydroxide, sodium citrate, hydrophilic surfactant as but not limited to polysorbate, sodium lauryl sulfate. Non-volatile solubilizer(s) of hydrophilic character includes also, PEO and its derivatives, cellulosic derivatives such as but not limited to HPMC having aqueous viscosity of Not More Than (NMT) 500 mPas (2%, 25 C) and HPC of low Molecular Weight (MW) (up to 95.000) having aqueous viscosity of Not More Than (NMT) 150 mPas (5%, 25 C). 3) volatile solubilizer(s) that evaporate under normal indoor atmospheric conditions of temperature and pressure, non-limiting examples include ketone derivatives such as methyl ethyl ketone and acetone, alcohols such as methanol and ethanol, and alkanes such as hexane.

The term “solubilizing system” A solubilizing system for oral films refers to a combination of ingredients or components within the formulation of the film that enhances the solubility and dissolution of hydrophobic or poorly water-soluble substances, such as active pharmaceutical ingredients (APIs) or bioactive agents. The solubilizing system is designed to improve the dispersion of these substances within the film matrix, ensuring their effective release and bioavailability when the film is administered orally. In the context of oral films for pets or humans, a solubilizing system may include various excipients, surfactants, co-solvents, and other additives that interact with the hydrophobic components, breaking down their structure and enabling them to mix more readily with the surrounding aqueous environment. This results in a homogenous and stable formulation that allows for efficient absorption of the active ingredients upon administration.

The purpose of a solubilizing system in oral films is to overcome the challenge of delivering poorly water-soluble compounds, which can otherwise lead to uneven distribution, reduced bioavailability, and compromised therapeutic effects. By using an effective solubilizing system, the oral film can enhance the solubility and dissolution of the bioactive agents, thereby optimizing their absorption and ensuring consistent and reliable therapeutic outcomes.

The term “mucoadhesive” and variations thereof generally refers to film matrix or pharmaceutical dosage forms interacting by means of adhesion with the mucus that covers epithelia.

The term “Antifoaming agent” refers to any substance added to prevent or counter the foam generation in the formulation. Generally, these agents have surface active properties and are insoluble in the foaming medium. Commonly used antifoaming agents are but not limited to certain alcohols (cetostearyl alcohol), insoluble oils (castor oil), polydimethylsiloxanes and other silicones derivatives, ether and glycols.

The term “mucoadhesive film former” refers to polymers that form the film matrix, film strip, film sheet and dissolve in the aqueous environment and result in bio-adhesive properties to the mucosa. Non-limiting examples include the following and their derivatives: PEO, Pullulan, CMC, HPC, HPMC, HEC, ethyl cellulose (EC), polyvinyl alcohol (PVA), Polymethacrylate polymers, including their derivatives. Examples of mucoadhesive materials that can be used to prepare the mucoadhesive film matrix are not limited to the following and their derivatives: poly(ethylene oxide), polyvinyl pyrrolidone, poly(acrylic acid) derivatives (e.g., commercially available Carbopol®), polycarbophil, polyoxyalkylene ethers, polymethacrylates, polymethacrylates-based copolymers (e.g., commercially available Eudragit®), biodegradable polymers such as poly(D,L-lactide-co-glycolide) (e.g., commercially available Resomer®), anionic biopolymers such as hyaluronic acid, or sodium carboxymethylcellulose, cationic biopolymers such as chitosan or poly(L-lysine) and other cellulose derivatives. Other mucoadhesive polymers that can be used include methyl vinyl ether-maleic acid, a mixed salt of sodium/calcium methyl vinyl ether-maleic acid, methyl vinyl ether-maleic anhydride, and half esters (monoethyl; monobutyl and isopropyl ester) of methyl vinyl ether-maleic anhydride copolymers (e.g., commercially available Gantrez®).

An agglomeration inhibitor in the context of oral films refers to a substance or ingredient that is added to the formulation of the film to prevent or minimize the formation of agglomerates or clusters of particles. Agglomerates are formed when small particles stick together, which can negatively impact the quality, uniformity, and effectiveness of the oral film. Agglomeration inhibitors help maintain the desired properties of the film, such as its texture, appearance, and the even distribution of active ingredients. Examples include butare not limited to the following and their derivatives: Polyvinylpyrrolidone and Hydroxypropylmethylcellulose (HPMC) where the polymer structure combines both hydrophobic (methoxy group) and hydrophilic substitutions (hydroxypropoxy group) and have aqueous viscosity up to 15000 mPas (2%, 20 C), employed alone or mixed with Methyl cellulose (MC) of aqueous viscosity up to 5040 mPas (2%, 20 C).

The term “stabilizer” refers to a molecule that prevent chemical degradation such as, D-a-tocopheryl polyethylene glycol succinate (TPGS), citric acid, vitamin E, ascorbic acid, Ethylenediaminetetraacetic acid (EDTA), glutathione, L-Cysteine, Tocobiol, BHT (butylated hydroxytoluene), BHA (beta hydroxy acid), or a combination thereof.

The term “crystal growth inhibitor” refers to polymers that inhibit or delay crystal growth in the nucleus comprising examples which are not limited to the following and their derivatives: poly(vinylpyrrolidone) (PVP), hydroxypropyl methylcellulose acetate succinate (HPMCAS), HPMC, HPC, MC, poly(vinylpyrrolidone)/vinyl acetate (PVP/VA) and poly(vinyl acetate) (PVAc).

The term “preservative” refers to an agent that extends the storage life of food and non-food products by retarding or preventing deterioration of flavor, odor, color, texture, appearance, nutritive value, or safety. A preservative need not provide a lethal, irreversible action resulting in partial or complete microbial cell destruction or incapacitation. Sterilants, sanitizers, disinfectants, sporicides, viracides and tuberculocidal agents provide such an irreversible mode of action, sometimes referred to as “bactericidal” action. In contrast, a preservative can provide an inhibitory or bacteriostatic action that is reversible, in that the target microbes can resume multiplication if the preservative is removed. The principal differences between a preservative and a sanitizer primarily involve mode of action (a preservative prevents growth rather than killing microorganisms) and exposure time (a preservative has days to months to act whereas a sanitizer has at most a few minutes to act). In specific embodiments, the preservative includes but is not limited to at least one of following or their derivatives such as sodium benzoate, methyl paraben, propyl paraben, and sodium sorbate.

The term “high loading” as used herein can describe a film with a high amount of API such as a film above 50 mg of API. High loading can also be described in terms of film composition such as up to 40% of the film being composed of an API.

The term “bioavailability” will have its meaning as prescribed in the art, as the ability of a drug or other substance to be absorbed and used by the body. Bioavailability is an important factor in oral film technology. The sublingual mucosa has high membrane permeability due to its thin membrane structure and high vascularization. Due to this rapid blood supply, it offers very good bioavailability. Enhanced systemic bioavailability is owing to skipping the first-pass effect and better permeability is owing to high blood flow and lymphatic circulation. In addition, the oral mucosa is a very effective and selective route of systemic drug delivery because of the large surface area and ease of application for absorption. In studies, thin films have shown their abilities such as improving the initial effect of the drug and duration of this effect, decreasing the frequency of dosing, and increasing the effectiveness of the drug.

The term “plasticizer” as used herein refers to a substance that produces or promotes plasticity and flexibility and reduces brittleness. Plasticizers can be advantageously employed in film formulations as needed to suitably modify the flexibility of the film to facilitate processing and allow the film to easily conform to the shape of the oral mucosa to which the film is applied. Plasticizers may reduce the glass-transition temperature of the film forming polymers (e.g., the water soluble polymer or water soluble polymers in the film). Examples of plasticizers that can be used in the disclosed oral film dosage forms include but are not limited to the following and their derivatives such as triacetin, triethyl citrate, tributyl citrate, acetyl tributyl citrate, acetyl triethyl citrate, trioctyl citrate, acetyl trioctyl citrate, trihexyl citrate, sorbitol, maltitol, dibutyl sebacate, PEG 300, PEG 400, Glycerine, etc. Plasticizer(s) may be added in an amount up to 25%, alone or as a combination, of the total mass of the film oral dosage form, such as from 0% to 25%, 1% to 20%, 2% to 15% or 5% to 10%.

The term “flavoring agent” or “flavor” and variations thereof generally refers to concentrated preparations, with or without flavor adjuncts required in their manufacture, used to impart flavor, with the exception of salt, sweet, or acid tastes. Flavoring agents may be classified as natural, artificial, or natural and artificial (N&A) by combining the all-natural and synthetic flavors or other forms known in the art. Flavoring agents are categorized by their physical classification as solid flavoring agents and liquid flavoring agents.

The term “natural flavors” and variations thereof generally refers to flavoring agents that come from natural sources such as a spices, fruits, or vegetables. They can also come from herbs, barks, roots, or similar plant materials. Natural flavors also come from meat, seafood, poultry, eggs, and dairy products. Flavors are only used to add taste to foods; they are not generally nutritional in their definition.

The term “artificial flavors” and variations thereof generally refers to flavoring agents that do not meet the definition of natural flavor. The chemical compositions of natural and artificial flavorings do not usually differ considerably; it is the source that differs. Active ingredients in natural flavors used to impart flavor are often identified and reconstructed synthetically with reasonable accuracy.

The term “natural & artificial (N&A) flavors” and variations thereof generally refers to natural flavors combined with synthetic ingredients to enhance flavor balance and fullness. These flavors are generally classified according to type and taste sensation.

The term “liquid flavor” and variations thereof generally refers to a flavor in liquid phase with or without liquid carrier. The texture is generally dependent on the solvent within which they are prepared. Liquid flavors are available both as oily (e.g., essential oils) or non-oily liquids. Examples of liquid flavor include but are not limited to essential oils, fluid extracts, tinctures, and distillates or other forms known in the art. The term “liquid” and variations thereof generally refers to viscous liquids, slurries, foams, pastes, gels and the like.

Other known techniques used to cover the perceived unpleasant taste of active agents include the addition of taste maskers, sweeteners and flavor enhancers (sometimes called “savor boosters”).

The term “Flavor Enhancer” and variations thereof generally refers to compounds that particularly enhance certain tastes or reduce undesirable flavors without having an especially strong taste of their own. They harmonize taste components and make food/drug preparations more palatable. Examples include but are not limited to maltol, ethyl maltol and monosodium glutamate, glutamic acid, glutamates, purine-5-ribonucleotides, inosine, guanosine, adenosine 5-monophosphates, sugars, sweetener, carboxylic acids (e.g., citric, malic, and tartaric), common salt (NaCl), amino acids, some amino acid derivatives (e.g., monosodium glutamate-MSG), and spices (e.g., peppers) are most often employed, yeast, yeast extract, dried yeast and others or mixtures thereof.

The term “Taste Masker” and variations thereof generally refers to an ingredient capable of covering or at least making more acceptable an unpleasant odor or taste in a food or pharmaceuticals. Of the many tastes that must be masked in pharmaceuticals, bitterness is most often encountered; to mask it completely is challenging. Examples of bitter maskers include but are not limited to licorice, coffee, chocolate, mint, grapefruit, cherry, peach, raspberry, orange, lemon, lime, advantame and others or mixtures thereof. Syrups of cinnamon, orange, citric acid, cherry, cocoa, wild cherry, raspberry, or glycyrrhiza elixir, raspberry and other fruit syrups can be used to effectively mask salty and bitter tastes in a number of drug products. Metallic tastes in oral liquid products (e.g., iron) are often masked by extracts of guarana, a tropical fruit, but can be masked by other extracts and agents.

The term “sweetener” and variations thereof generally refers to a solid or liquid ingredient that is used to impart a sweet taste to food or drug product. Sweeteners are often classified as either nutritive (caloric) or non-nutritive (non-caloric), natural or synthetic. Examples of sweeteners include but are not limited to sucrose, dextrose, lactose, glucose, advantame, sorbitol, mannitol, liquid glucose, honey molasses, saccharin, sucralose, rebaudioside A stevia, rebaudioside M stevia, stevioside, mogroside IV, mogroside V, alitame, saccharin, neohesperidin dihydrochalcone, cyclamate, neotame, N-[3(3-hydroxy-4-methoxybenzyl yl) propyl]-L-α-aspartyl]-L-phenylalanine 1-methyl ester, N-[3-(3-hydroxy-4-methoxyphenyl)-3-methylbutan yl]-L-α-aspartyl]-L-phenylalanine 1-methyl ester, N-[3-(3-methoxy-4-hydroxyphenyl) propyl]-L-α-aspartyl]-L-phenylalanine 1-methyl ester, curculin, cyclamate, aspartame, acesulfame potassium and others or mixtures thereof.

The term “colorants” and variations thereof generally refers to any dye, pigment, or other substance made by a process of synthesis or similar artifice, or extracted, isolated, or otherwise derived, with or without intermediate or final change of identity, from a vegetable, animal, mineral or other source and that, when added or applied, can impart color to a food, drug, or cosmetic or to the human body. Color makes products attractive, appealing, appetizing, and informative. Examples of colorants include but are not limited to Brilliant blue FCF, Indigotine, Alphazurine FG, Indigo, Indanthrene blue, Resorcin brown, Fast green FCF, Alizarin cyanine green F, Quinizarine green SS, Pyranine, Dibromofluorescein, Diiodofluorescein, Erythrosine yellowish Na, Copper phthalocyanine, Erythrosine, Ponceau SX, Lithol rubin B, Lithol rubin B Ca, Toney red, Tetrabromo fluorescein, Eosine, Tetrachlorotetra-bromofluorescein, Yellow Iron Oxide, Ultramarine Blue, Zinc Ferrite, Chromium Oxide Green, Titanium Dioxide, Zinc Oxide, Phloxine, Helindone pink CN, Brilliant lake red R, Acid fuchsine, Lake bordeaux B, Flaming red, Alba red, Allura red AC, Alizurol purple SS, Alizarin violet, Tartrazine, Sunset yellow FCF, Fluorescein, Napthol yellow S, Uranine, Quinoline yellow WS, Quinoline yellow SS and others or mixtures thereof.

The terms “permeation enhancer” and variations thereof generally refers to a chemical compound which is added into the formulation along with the target drug in order to improve active agent permeation through the biological membrane such as the skin, nasal, oral and intestinal mucosae. Examples comprising but not limited to bile salts, benzene derivatives, fatty acids and derivatives such as oleic acid and lauric acid, glycerides such as phospholipids and medium chain glycerides, surfactants such as sodium lauryl sulphate and polysorbates, inclusion complexes such as cyclodextrins derivatives, pH modulators, chelators such as Disodium ethylene diamine tetra acetate, D-a-tocopheryl polyethylene glycol succinate (TPGS) and mucoadhesive excipients such as chitosan and polycarbophil.

The terms “permeation co-enhancer” and variations thereof generally refers to excipient used to enhance the action of the permeation enhancer and therefore improves API permeation by decreasing the lag time between the administration of permeation enhancer and the enhancement of permeation. Examples comprising but not limited to polyethylene glycols, propylene glycol, and glycerol.

The “surface pH” is the pH measured on a surface of the film, such as the top or bottom surface of a monolayer film or on an exposed surface of the layer containing the active in a multilayer oral film. The film is prepared for pH testing by slightly wetting the film (adding water as needed for a pH test—e.g. one to three drops). The pH is then measured by bringing the electrode in contact with the surface of the oral film. This measurement of the surface pH is preferably performed on several films of the same formulation.

The terms “blend” or “blending media” and variations thereof generally refers to the combination of the oral film formulation with the presence of solvents.

The term “drug absorption” or “absorption” as used in this specification, refers to the process of movement from the site of administration of a drug toward the systemic circulation, e.g., into the bloodstream of a subject.

The term “residence time” as used in the specification refers to the time taken by the film to disintegrate on the sublingual or buccal mucosa.

Preferred film dosage forms include sublingual and buccal film oral dosage forms. Buccal and/or sublingual mucosa absorption allows the drug to be absorbed directly into the blood stream skipping the hepatic metabolism. From a pharmaceutical formulation perspective this is particularly challenging, as the process of transmucosal permeation needs to be carefully optimized to obtain an acceptable pharmacokinetic profile. The use of a long lasting oral film allowing the dissolving film to direct the active agent through the mucosa directly to the blood stream may be desired to improve the absorption profile of the API and consequently improve bioavailability.

The buccal or sublingual film dosage form can comprise a single film layer, or multiple layers. In some embodiments, a bilayer or multilayer film would include a mucoadhesive layer containing the API which is placed against the oral mucosa and a second layer directed outwards from the mucosa serving as a protective barrier against abrasion from the tongue or mastication or simply against constant washing of the saliva. This protective layer also serves to favor the directed absorption of the API within the oral cavity rather than enteric uptake in the gastrointestinal (GI) tract.

As used herein, the term “animals” is meant to indicate mammals, and to exclude humans.

The present disclosure provides methods and products for locally administering one or more active agents via adhesion of a film to a mucosae membrane such as, for example, mucosae membrane included in a human or non-human mammalian oral cavity. A dissolving film containing an active agent is placed upon a mucosae membrane, such as a membrane within the oral cavity. The hydrophilic nature of the dissolving film causes the film to stick to the mucous membrane.

In order to enhance the acceptability of the delivery systems of the present disclosure for animal subjects, certain embodiments comprise flavoring agents such as but not limited to beef, chicken, liver, bacon, cheese, apple, smoke, fish, mint such as spearmint or peppermint, and combinations thereof and others.

Maropitant, belonging to the class of organic compounds diphenylmethanes, a substituted quinuclidine having the chemical name (2S,3S)-(Diphenylmethyl)-N-[2-methoxy-5-(2-methyl-2-propanyl)benzyl]quinuclidin-3-amine, the systematic (IUPAC) name being (2S,3S)—N-(5-tert-Butyl-2-methoxybenzyl)-2-(diphenylmethyl)-1-azabicyclo[2.2.2]octan-3-amine, is a neurokinin (NK1) receptor antagonist that blocks the pharmacological action of substance P in the central nervous system (CNS). Maropitant is used in the prevention and treatment of vomiting in animals.

Maropitant also has mild pain-relieving, anti-anxiety and anti-inflammatory effects. Maropitant and similar classes of compounds can be used pharmaceutically as antiemetics, autonomic agents, benzene derivatives, central nervous system agents, gastrointestinal agents and peripheral nervous system agents.

Regarding injectable dosage forms, the users (pet owners) or the patients (pets) must refer to professional health and veterinary care for dose administration. In parallel, injectable dosage forms are not the most preferable due to pain and local irritation, especially for chronic treatment where repeated and more frequent administration is necessary. The users and patients preferable path is the oral route since the gastrointestinal tract has a higher surface for absorption. However, several limitations such as the hepatic first pass mechanisms as well as gastric degradation of a drug make emerging advances in this area challenging for drug bioavailability.

It is known in the art that an increase of API loading in the oral dosage form will boost bioavailability and will compensate for metabolism and the portion of the API lost through excretion, however this approach might increase the potential risk of toxicity, and the unbalanced risk versus benefit must be addressed.

Drugs for veterinary use may require higher dosage depending on the animal's weight, which translates to a higher dosage per tablet. A higher dosage tablet implies a tablet of higher dimensions to effectively carry a high amount of API and excipients.

The administration of tablets to an animal is challenging since animal are likely to attempt to expel the tablet from its mouth, and therefore adherence becomes an issue. Often, pet owners will break the tablet into pieces and hide them inside the animal's food in order to mask the bad taste and attempt to ensure that the entire dose is consumed. While tablets may be broken into smaller pieces or even crushed as a means of overcoming swallowing difficulties or adherence, this is not a suitable solution for many tablet or pill forms. For example, crushing or destroying the tablet or pill form to facilitate ingestion, alone or in a mixture with food, may destroy the dosage form's controlled release properties, taste masking properties, or otherwise affect the pharmacokinetic properties of the drug.

Oral films are an alternative dosage form to replace the tablet owning several benefits such as a quicker onset of action and better drug bioavailability. However, due to the limited size, weight and thickness of the film, it cannot easily accommodate higher drug loading. Films of a bigger size may not fit in the animal's mouth, and films of a greater thickness will have unacceptable disintegration time or mouth feel, all of those issues will bring back to square one as the tablet.

The use of a mucoadhesive matrix will improve the ease of administration and the compliance for therapy by reducing the risk of the animal spitting out the dosage form by creating an intimate contact with the absorption site to increase likelihood of effective permeability. However, with the presence of cationic drugs, the anionic mucoadhesive excipient are non-compatible since there is a risk of ionic complex formation resulting in formulation failure. On the other side, the cationic mucoadhesive excipient will require a stronger acidic microenvironment for their solubilisation which could be an irritant to the buccal mucosa and will be neutralized by the saliva pH. Use of non-ionic polymers with improved wettability help adhesion by hydration and formation of hydrogen bonds among the functional groups of the polymers and mucosal layer.

Providing an oral film where the API is suspended will have the advantage of being less prone to drug recrystallization, but may make the solubilisation kinetic of the API and drug absorption slower due to a short residence time and limited saliva volume for solubilization. Combining approaches of suspension and solubilization allows for a dual system—the solubilized part boosts the system to cover the first portion of the drug for permeability since the suspended API has slower drug absorption (amorphous molecules are generally more absorbable than a crystalline form); the suspended API and/or the remaining portion of the solubilized part will promote continuity of API absorption even in the buccal area and or the gastric duct.

An oral film containing a solubilized API delivers the active agent more quickly, thereby increasing buccal and/or gastric absorption, however there is a risk of API recrystallization. Selection of excipients is important here to prevent such instability. However, in some embodiments, to maintain a soluble API, generally a ratio of less than 1:1 API to solubilizer is required and this results in films of higher weight and greater dimensions.

The use of an organic solvent to solubilize the API(s) are known in the art but due to limited film forming polymer solubility, choice of polymer is limited. Since the solvents cannot be maintained at a higher percentage after film drying for safety reasons (ICH guidelines), the API is more prone to recrystallization after drying.

Regarding high API loading, the requirement to use a higher ratio of crystal inhibitor such as poly(vinylpyrrolidone) (PVP), hydroxypropyl methylcellulose acetate succinate (HPMCAS), poly(vinylpyrrolidone)/vinyl acetate (PVP/VA) and poly(vinyl acetate) (PVAc) makes the percentage of solids higher in the film formulation.

Another alternative for volatile solubilizer(s) of the API are the non-volatile solubilizer(s) which remain in the film after the drying step but in addition to increasing the solid percentage, such solubilizer(s) are usually liquids having a more lipophilic character which results in formulations showing slower drug dissolution and lower API permeation, in the absence of a permeation co-enhancer. The use of liquid solubilizer(s) such as fatty acids, oils or other lipid bases can generate films with physical instability, oily aspects, longer disintegration time, drug precipitation, phase separation in the film blend and low permeation profile. To mitigate the low permeation issue, a permeation co-enhancer can be used.

During a permeability study using Franz Cell equipment and buccal pig mucosa membrane, a suspended maropitant film prototype made in aqueous neutral pH has a long lag time and low permeation profile. The in-vivo results of the later film prototype were not fully satisfactory. A faster and increased extension of absorption was required.

Maropitant film prototypes where the API is solubilized in organic solvent became crystalized, even with the addition of a crystal growth inhibitor (due to the high loaded API), resulting in no permeation through the pig mucosa. Maropitant, a cationic drug at neutral pH and precipitate was formed with polyacid polymer(s) such as CMC, Carbomer and polycarbophil.

Films made with a chitosan polymer did not disintegrate in aqueous neutral pH since Chitosan is a polycationic biopolymer that can be protonated only in weak organic acid solutions with pH lower than its pKa (˜6.3-6.5).

Maropitant oral film made with oleic acid at more than 10% showed oily aspects, longer disintegration time in neutral pH, and long lag time during the permeation study.

Maropitant oral film made with MCT oil in MEK (methyl ethyl ketone) showed oily aspects and drug crystallization.

The inventors discovered that making the entire API of high strength film permeable through the buccal mucosa will require solubilized API in a mucoadhesive matrix with longer residence time. It was observed that an oral film was needed that further improved the bioavailability by increasing the solubility or presence of amorphous API, improving the permeation in buccal mucosa to limit first hepatic pass, and formulating a dosage form that is highly mucoadhesive to allow increased contact of the API with the mucosa, for better compliance, alternative administration procedure and reproducible dosing.

Further improvement of API solubilisation will confer a higher surface area of the API, provide the API in an amorphous state, and consequently trigger an increased rate and extent of absorption of the API in the buccal microenvironment, which reduces the lag time for drug buccal permeation. In addition, once the film is applied on the mucosa, the films dissolve upon contact with a wet surface of the mucosa. When pH modulator(s) and or/the other components of the oral film become hydrated they create a micro-environment pH, the pH of which is predicted with the measurement of the film surface pH. The said micro-environment pH helps API ionization and creates hydrogen bonding with water molecules and therefore said API solubilization. The soluble ionic API will be in an equilibrium with soluble non ionic form of the API that is favorable for the API absorption through the mucosa. As soon as the soluble non ionic form is consumed by permeation through the mucosa, the equilibrium will shift into the direction of providing more soluble non ionic form of the API. Favorable micro-environment pH for basic API is below its pKa, and for acidic API it will be above its pKa.

Furthermore, providing film matrices with optimal physical characteristics such as physical appearance, folding endurance, tensile strength and percentage elongation, allows maintenance of the performance of the product during its shelf life, which is important. Therefore, a combination of a matrix system using solubilized API in non-volatile solubilizer(s) and suspended API in polymeric mucoadhesive film matrix makes the conception of higher loading film feasible with optimal permeability, film size, stability and flexibility. The said optimal film will have polymers with improved wettability that help mucoadhesion by fast hydration. When applied to the buccal mucosa, the readily solubilized API part boosts the permeability and reduces the lag time of permeation, while the suspended API part and/or the remaining solubilized part will promote continuity of the absorption even in the buccal and or in the gastrointestinal tract.

The present disclosure provides for a stable mucoadhesive flexible film of improved bioavailability, higher strength dosage and shorter lag time for permeation.

### Dual System: Solubilization and Suspension

The film matrix of a high strength API succeeds only when two different approaches are combined: a solubilization system using emulsion providing amorphous API, and a suspension system providing finely dispersed crystals. When only one system is used (emulsion or suspension), the film matrix fails to garner the desired properties for the high strength API or is unachievable.

In a preferred embodiment, the active pharmaceutical ingredient (API) is present in the film matrix into two ways, solubilized and suspended to make film of high strength API feasible and successful. The solubilized API increases the API surface area and prepares the API to be released as an amorphous form; giving the highest dispersion and providing the quickest onset of absorption while overcoming the dissolution rate limit. This allows for the quickest onset of action and an improved bioavailability.

In order to solubilize a poorly water soluble API, at least one solubilizer is used. Said solubilizer(s) is preferably non-volatile, and aims to maintain the amorphous form available in the dry film.

Many difficulties arise when formulating a high-loading film with only solubilized API due to the required amount of the solubilizer(s) and optionally crystal growth inhibitor polymer(s) resulting in a film of higher weight and unacceptable thickness. The use of the only-suspended-API approach uses less solids in the film since it does not require such an amount of solubilizer(s) and stabilizer(s) as in the solubilized prototype (crystal growth inhibitor polymer), but requires a small amount of viscosity increasing agent/suspending agent ranging from one 10^{th} to one 15^{th} the amount of API to maintain the stability of the dispersion. The suspended API has reduced permeability capability compare to the solubilized approach because the API release is dissolution-rate-limited, but has an increased surface area that facilitates the drug's dispersability and dissolution compared to physical mixtures and pure API. The suspended approach prevents or at least mitigates uncontrolled, undesired API recrystallization in the cast oral film that may generate different profiles of drug release and potentially resulting in product failure.

Combining the two approaches, solubilized and suspended API will allow the use of less total solids, allowing a design-stable oral film that includes a solubilized portion of API. The solubilized API will promote the fast drug release and permeation close to the biological membrane upon contact with the biological fluids in the body, while the suspended API will provide continuous concentrated API close to the biological membrane to maintain the gradient of concentration from the outer cell to the inner cell and promote API absorption.

The higher the percentage of API in the matrix, the closer the API molecules are and the more prone the API is to creating nucleus for recrystallization. As there is a limitation in terms of weight per surface area that can be coated, then the API should be limited to prevent the molecules from getting too close, unless there is a crystal growth inhibitor to prevent it, but the effect is limited and also increases the film weight.

To solubilize the API, a solubilizer/permeation enhancer of log p not more than 8 is chosen for this embodiment, because it provides better solubilisation power as per the maxim “like dissolves like”. This method allows the use of less than 6 parts of the said solubilizer with 1 part of low water-soluble API. The lipophilic character of the API and solubilizer/permeation enhancer is proportional to its log p and when it is higher (log p), this results in a film matrix that is fatty, and thus counters the film's wettability. The selected solubilizer will have permeation-enhancing properties which is key for drug absorption. By having permeation-enhancing properties, it is advantageous to reduce the amount of solids in the film formula.

Since the said solubilizer/permeation enhancer is lipid-based, it has low water solubility, which is inconvenient for drug release due to the decreased film wettability and hydration resulting in a longer lag time for permeation. To address those limitations, it is preferable not to exceed 5% of the said solubilizer/permeation enhancer of lipophilic character to limit the lipophilic character of the film matrix, switch the said solubilizer/permeation enhancer to a water soluble solubilizer/permeation enhancer of low log p or combine both type of solubilizers/permeation enhancers (the one of lipophilic character with the one of the water soluble character) to boost the film hydration and wettability and to increase the amount of solubilized API per film when using higher ratios of combined solubilizer/permeation enhancers.

In addition, adding a surfactant system that meets the required HLB for the API and the lipophilic solubilizer/permeation enhancer will enhance the wettability of the film in aqueous media, help with the drug's dispensability, film dissolution, drug release and permeation.

Furthermore, a suspending agent (also referred to as a “viscosity increasing agent”) is added to prevent adjacent suspended particles from coming close enough to join each other and promoting recrystallization. Sufficiently increasing the viscosity of the drug vehicle (polymer matrix) enables steric stabilization of the dispersed drug. In addition to preventing the drug from crystalizing, the suspending agent thickens and stabilizes the emulsion containing the API through increased viscosity. Additionally, the said suspending agent interacts with biological mucosa to create and strengthen the oral film mucoadhesion, which increases drug permeation while maintaining the proximity of the API close to the site of absorption.

To assist API permeation and reduce lag time caused by the lipophilic solubilizer/permeation enhancer, a permeation co-enhancer is used to increase the aqueous solubility of the said solubilizer/permeation enhancer which allows it to leave the vehicle more readily permitting the said solubilizer/permeation enhancer to enhance membrane fluidity and allowing for a greater likelihood of drug permeation.

After the preparation of the active pharmaceutical phase (API, solubilizer(s) with surfactant(s) system, permeation co-enhancer and suspending agent/viscosity increasing agent), a low viscosity polymer of solubility enhancing properties is added to the blend, together with the said phase to assist incorporation of the organic API in the aqueous system, thereby preventing further precipitation of the API in the dry film.

To flavor the film, the flavor, flavor enhancer, and sweetener(s) are blended with the wet blend to create an acceptable flavor profile in the final dry to improve film palatability and mask or eliminate any potential aftertastes, while also intensifying sweetness, extending sweetness and enhancing other flavors.

During the last step of the blending process, polymer(s) making a film-forming matrix are added, at least one having quick wettability to create a mucoadhesive matrix together with another one that prevents API crystal growth in the final dry film.

The present disclosure relates to providing a film forming matrix carrying a high loading of a drug substance where a considerable portion is intended to be released and absorbed buccally to avoid hepatic first pass and resulting in better API bioavailability.

In one embodiment, the film forming matrix includes an API dissolved (amorphous) using solubilizers with appropriate Log P (log p not more than 8) at ratio of API to solubilizer ranging from 10:3 to 10:60, and a suspended API to increase loading and maintain film weight low.

The high loading film has a portion of the API solubilized which represents more than one tenth of the entire dose, the said solubilized API is maintained as an amorphous form in a surfactant system that assists in preventing solubilization and dispersibility of the said API. The other portion of the API is suspended to make a film forming matrix of acceptable weight and dimensions. The concept is to carry and release a soluble API (amorphous) as uncharged and of high dispersibility close the site of absorption of biological mucosa resulting in an early onset of action that will be maintained later by the release and absorption of the API from a suspended portion of the API.

In a preferred embodiment, the film forming matrix includes an API dissolved (amorphous) using solubilizers with appropriate Log P (log p not more than 8) at ratio of API to solubilizer ranging from 1:1 to 1:2.5, and a suspended API to increase loading and maintain film weight low.

A preferred embodiment also includes the presence of a lipophilic solubilizer/permeation enhancer to help to carry the amorphous form of API, more soluble than the API crystal form, in the film and consequently boost the film hydration and wettability.

A preferred embodiment also includes a suspended API under conditions that will potentially be able to be solubilized for addition buccal absorption.

A preferred embodiment includes a permeation enhancer which preconditions the mucosa cells membrane to help increasing membrane fluidity, the cell membrane of the mucosa will be continuously fed in the beginning with amorphous API of better solubility than the crystal one. It further includes mucoadhesive polymers that help for maintaining an intimate contact of the film with the biological mucosa for acceptable period of time, and a suspending agent to prevent adjacent suspended particles from coming close enough to join each other and promoting recrystallization, in addition a solubility enhancer is added to solubilize the API and imped its recrystallization.

The concept herein is to initiate a high onset of action by allowing an early release of the API in an amorphous state from the mucoadhesive film forming matrix. The API is readily soluble when exposed to biologic fluid and is introduced in an oral film in proximity with buccal mucosa of increased cell membrane fluidity to promote its permeation. The said matrix prevents crystallization and high dispersible particles where film forming polymers make the residence time in the buccal site favorable to increase the chance for higher drug bioavailability. The oral film provides said API in high loading to cover higher subject weight and to maintain concentration gradients to promote drug absorption. The said high strength oral film has a second portion of API provided as suspended API to make a back-up for the continuity of drug release and by consequence the drug action.

In general, an oral film dosage form includes solubilized API in solubilizer, permeation co-enhancer, suspended API, a surfactant system, a suspending agent/viscosity increasing agent and polymeric film matrix.

In general, an oral film dosage form includes solubilized API in solubilizer, permeation enhancer, permeation co-enhancer, suspended API, a surfactant system, a suspending agent/viscosity increasing agent, a plasticizer, a flavor system, a chemical stabilizer and polymeric film matrix.

In certain embodiments, an oral film dosage form comprises high strength of API, solubilizer, permeation co-enhancer, suspending agent/viscosity increasing agent, a mucoadhesive film former polymer, an amphiphilic solubility enhancer, an agglomeration inhibitor and a surfactant, wherein a portion of the API is solubilized is less than the remaining suspended part. The total API content per film is at least 50 mg representing at least 22% of the total dry weight of the oral film.

According to some aspects of the disclosure, the oral film dosage form comprises at least one API in a dual system: API in solubilizer(s) and suspended API in polymeric mucoadhesive film matrix.

According to some aspects of the disclosure the solubilized API represents more than one tenth of the entire dose.

According to some aspects of the disclosure, a multi-layer oral film made of a portion of Maropitant or a salt thereof is solubilized and made in a separate film layer than the Maropitant or a salt thereof that are suspended.

According to some aspects of the disclosure, the oral film dosage form contains a solubilizer present in amounts of at least 4% and not more than 30% of the total composition of the oral film.

According to some aspects of the disclosure, the solubilizer is lipid-based and is present in amounts up to 10% of the total composition of the oral film dosage form, and is made of: one or more than one fatty acid, derivatives thereof or combinations thereof and preferably oleic acid or caprylic acid.

According to some aspects of the disclosure, the solubilizer is lipid-based and preferably is made of one or the combination of the following: oleic acid derivative or caprylic acid derivative.

According to some aspects of the disclosure, the solubilizer is a benzene derivative and is present in amounts up to 25% of the total composition of the oral film dosage form, and is preferably benzyl alcohol or benzyl acetate.

According to some aspects of the disclosure, the solubilizer has a log p not more than 8.

According to some aspects of the disclosure, the oral film dosage form contains a solubilized API in solubilizer system.

According to some aspects of the disclosure, the oral film dosage form contains a solubilized API in solubilizer system. Said solubilizer system contains at least one solubilizer.

According to some aspects of the disclosure, the oral film dosage form contains preferably a solubilizer system that includes solubilizer with permeation-enhancing properties.

According to some aspects of the disclosure, the solubilized API to solubilizer(s) ratio is from 10:3 to 10:60. According to some aspects of the disclosure, the solubilized API in solubilizer system made of a combination of the following: 10 parts of lipid based solubilizer(s) and not more than 13 parts benzene derivative solubilizer(s).

According to some aspects of the disclosure, the solubilized API in solubilizer system is made of a combination of the following: 1 part of hydrophilic surfactant(s) and not more than 4 parts of benzene derivative solubilizer(s).

According to some aspects of the disclosure, the solubilized API in solubilizer system is made of a combination of the following: 10 to 12 parts of lipid based solubilizer(s) and 5 to 15 parts of benzene derivatives.

According to some aspects of the disclosure, the solubilized API in solubilizer system is made of a combination of the following: 10 parts of lipid based solubilizer(s) and not more than 15 parts of a non-volatile solubilizer with pH modulator function.

According to some aspects of the disclosure, the solubilized API in solubilizer system is made of a combination of the following: 10 parts of lipid based solubilizer(s), not more than 12 parts of a non-volatile solubilizer(s) with pH modulator function and not more than 6 part of hydrophilic surfactant(s).

According to some aspects of the disclosure, the solubilized API in solubilizer system is made of a combination of the following: 5 parts of lipid based solubilizer(s) and not more than 7 parts benzene derivatives and not more than 8 parts of non-volatile solubilizer with pH modulator function.

According to some aspects of the disclosure, the oral film dosage form includes a water-soluble API.

According to some aspects of the disclosure, the oral film dosage form has at least one API without ionisable group(s).

According to some aspects of the disclosure, the oral film dosage form has at least one API having at least one ionisable group.

According to some aspects of the disclosure, the oral film dosage form has an API of acidic character such as aspirin or ascorbic acid.

According to some aspects of the disclosure API is of basic character such as but not limited to loxapine, maropitant, dimethyltryptamine (DMT) or rizatriptan.

According to some aspects of the disclosure, the oral film dosage form has at least one API of amphoteric character such as diclofenac.

According to some aspects of the disclosure, the oral film comprises at least one pH modulator.

According to some aspects of the disclosure, the oral film has a non-volatile solubilizer with pH modulator function at not more than 15% total dry weight of the oral film.

According to some aspects of the disclosure, the pH modulator(s) creates a favorable pH for API solubilisation, maintains a portion of the drug un-ionized.

According to some aspects of the disclosure, the pH modulator is HCl, succinic acid, citric acid, sodium hydroxide, sodium citrate, sodium benzoate or combinations thereof.

According to some aspects of the disclosure, pH modulator(s) includes a buffer system comprised of citric acid/sodium citrate or benzoic acid/sodium benzoate, or combinations thereof.

According to some aspects of the disclosure, the preferred pH modulator is selected from the group of inorganic acid such as but not limited to hydrochloric acid, organic acid such as but not limited to citric acid, sodium citrate, succinic acid, and sodium succinate, derivatives thereof or combinations thereof.

According to some aspects of the disclosure, the pH modulator comprises no more than 12% of total dry weight of the oral film.

According to some aspects of the disclosure, the oral film comprises at least one, permeation co-enhancer is not more than 20% total dry weight of the oral film.

According to some aspects of the disclosure, the permeation co-enhancer is selected from the group of PEG 300, PEG 400, glycerol, propylene glycol, derivatives thereof or combinations thereof and is preferably polyethylene glycol 300.

According to some aspects of the disclosure, the permeation co-enhancer is not more than 10% of total dry weight of the oral film.

According to some aspects of the disclosure, the solubility enhancer is not more than 10% total dry weight of the oral film.

According to some aspects of the disclosure, the solubility enhancer is amphiphilic and is selected from the group of HPC SSL and poloxamer, derivatives thereof or combinations thereof.

According to some aspects of the disclosure, solubility enhancer is non-amphiphilic and is selected from the group of PEG, PEG derivatives, Medium chain triglycerides, glycerides derivatives or combinations thereof.

According to some aspects of the disclosure, the solubility enhancer is preferably hydroxypropyl cellulose SSL.

According to some aspects of the disclosure, the solubility enhancer is not more than 6% of total dry weight of the oral film.

According to some aspects of the disclosure surfactant system at not more than 14% of total dry weight of the oral film, and is made up of components selected from but not limited to polysorbate, labrafil M2125CS, labrasol, propylene glycol monocaprylate type I, propylene glycol monocaprylate type II, lecithin, hydroxylated lecithin, lecithin derivative and gelucire 44/14, derivatives thereof or combinations thereof.

According to some aspects of the disclosure, the surfactant system is preferably a combination of low HLB surfactant(s) (HLB up to 12) such as labrafil M2125CS and high HLB surfactant(s) (HLB equal to 12 and above) such as polysorbate 20.

According to some aspects of the disclosure, the surfactant system comprises preferably one surfactant such as lecithin, hydroxylated lecithin or lecithin derivative.

According to some aspects of the disclosure, the surfactant system is preferably a combination of 8 to 9 parts lecithin derivative with 1 to 2 parts of surfactant(s).

According to some aspects of the disclosure, the surfactant system is not more than 15% of total dry weight of the oral film.

According to some aspects of the disclosure, the surfactant system is preferably not more than 11% of total dry weight of the oral film.

According to some aspects of the disclosure, non-ionic polymers are formulated with a basic API.

According to some aspects of the disclosure, anionic polymers are formulated with an acidic API selected from the group of cmc and carbopol for mucoadhesion, whereas carbopol is solubilized at neutral pH as is the acid API.

According to some aspects of the disclosure, the oral film further comprises a suspending agent(s)/viscosity increasing agent(s) at not more than 15% and preferably not more than 6% of total dry weight of the oral film.

According to some aspects of the disclosure, the oral film further comprises a suspending agent(s)/viscosity increasing agent(s) at not more than 3%.

According to some aspects of the disclosure, the suspending agent(s)/viscosity increasing agent(s) is selected from the group of propylene glycol alginate, hydroxyl propyl methyl cellulose 4000, gellan gum, hydroxyethyl cellulose, guar gum, hydrolyzed guar gum, derivatives thereof, polysaccharide derivative, cellulose derivative or combinations thereof.

According to some aspects of the disclosure, the suspending agent/viscosity increasing agent is not more than 11% of total dry weight of the oral film.

According to some aspects of the disclosure, the oral film further comprises at least one plasticizer.

According to some aspects of the disclosure, the plasticizer is not more than 15% and preferably not more than 10% of total dry weight of the oral film.

According to some aspects of the disclosure, the plasticizer is selected from the group of PEG 300, PEG 400, glycerol, propylene glycol, triacetin, and is preferably polyethylene glycol and/or its derivative(s).

According to some aspects of the disclosure, the plasticizer is selected from the group of PEG 300, PEG 400, glycerol, propylene glycol, triacetin, triethyl citrate, derivatives thereof or combinations thereof.

According to certain aspects of this disclosure, the film forming matrix comprises at least one polymer selected from the group of: pullulan, polyvinyl pyrrolidone, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, ethylene oxide-propylene oxide co-polymers, collagen, albumin, poly-amino acids, polysaccharides, chitin, chitosan, carboxymethyl cellulose, derivatives thereof or combinations thereof.

According to certain aspects of this disclosure, the film forming matrix is preferably comprising at least one polymer selected from Poly(ethylene oxide), MW 200,000 (PEO 200000), hydroxypropyl cellulose (HPC LF) and Hydroxypropyl Methylcellulose (HPMC E50).

According to certain aspects of this disclosure, the film forming matrix comprises not more than 60% of the dry weight of the oral film, and preferably ranging from 25% to 50% of total dry weight of the oral film.

According to certain aspects of this disclosure, the film forming matrix comprises preferably 30% to 45% of total dry weight of the oral film.

In certain aspects of this disclosure, the disclosed formulations and excipients are specifically adapted for use in animals.

In certain aspects of this disclosure, the disclosed formulations and excipients are specifically adapted for use in animals, specifically to treat anti-emesis.

In certain aspects of this disclosure, the disclosed formulations further comprise flavor(s) to improve palatability for humans and animals.

In certain aspects of this disclosure, the disclosed formulations further comprise flavors and flavor enhancers to improve palatability for humans and animals.

In certain aspects of this disclosure, the disclosed formulations further comprise flavor(s), flavor enhancer(s), bitterness masker(s) and taste masker(s) to improve palatability for humans and animals.

According to some aspects of the disclosure, the API delivered by the oral dosage form is Maropitant.

According to some aspects of the disclosure, the API delivered by the oral dosage form is Maropitant Citrate.

According to some aspects of the disclosure, the API delivered by the oral dosage form is Maropitant salt.

According to some aspects of the disclosure, the API delivered by the oral dosage form is Ondansetron.

According to some aspects of the disclosure, the API delivered by the oral dosage form is Metoclopramide.

According to some aspects of the disclosure, the API delivered by the oral dosage form is Dolasetron.

According to some aspects of the disclosure, the API delivered by the oral dosage form is Aprepitant.

According to some aspects of the disclosure, the API is delivered with ginger extract to further treat anti-emesis.

According to some aspects of the disclosure, content of the API per oral film is at least 50 mg representing at least 22% of the total dry weight of the oral film and not more than 70% of the said total dry weight.

According to some aspects of the disclosure, the oral film has a surface pH ranging from 2.95 to pH 9.0, and more preferably a pH favorable to maintain a portion of drug un-ionized.

According to some aspects of the disclosure, the oral film further comprises a sweetener.

In certain aspects of this disclosure, the disclosed formulations further comprise flavor(s), flavor enhancer(s), taste masker(s) and sweetener to improve palatability for humans and animals.

According to some aspects of the disclosure, the oral film further comprises a colorant.

In specific embodiments, the oral dissolvable film is palatable to humans.

In specific embodiments, the oral dissolvable film is palatable to animals.

In specific embodiments, the external surfaces of the oral dissolvable film have a smooth texture.

In specific embodiments, the oral dissolvable film has a high tensile strength.

In specific embodiments, the oral dissolvable film is pliable.

In specific embodiments, the oral dissolvable film is non-sticky to touch.

In specific embodiments, the oral dissolvable film does not readily stick to another oral dissolvable film.

In specific embodiments, the oral dissolvable film is relatively soft to touch.

In specific embodiments, the oral dissolvable film has a chewable configuration.

In specific embodiments, the oral dissolvable film has a resilient configuration.

In specific embodiments, the oral dissolvable film has an elastic or malleable configuration.

In specific embodiments, the oral dissolvable film has a ductile property.

In specific embodiments, the oral dissolvable film further includes a bitter blocker.

In specific embodiments, the oral dissolvable film further includes a powder coating present on at least one external surface of the oral dissolvable film.

In specific embodiments, the oral dissolvable film further includes a powder coating present on two opposing external surfaces of the oral dissolvable film.

In specific embodiments, the method of preparing an oral dissolvable film is carried out in the order indicated.

In specific embodiments, each of the steps of the method of preparing an oral dissolvable film is carried out in the order indicated.

In specific embodiments, the mixing includes blending.

In specific embodiments, each of the multiple oral dissolvable films of the system independently has a dimension of at least 2 cm².

In specific embodiments, each of the multiple oral dissolvable films of the system independently has a dimension of up to 18 cm².

In specific embodiments, each of the multiple oral dissolvable films of the system independently has a dimension of 9 cm², ±2

In specific embodiments, each of the multiple oral dissolvable films of the system independently has a dimension of 9 cm², ±1.

In specific embodiments, each of the multiple oral dissolvable films of the system independently has a thickness of about 0.01 mm to about 2 mm.

In specific embodiments, each of the multiple oral dissolvable films of the system independently has a thickness of at least about 0.01 mm.

In specific embodiments, each of the multiple oral dissolvable films of the system independently has a thickness of up to about 2 mm.

In specific embodiments, each of the multiple oral dissolvable films of the system independently has a thickness of about 0.03 mm to about 1 mm.

In specific embodiments, some of the excipients used in the oral dissolvable films of the present disclosure may have one or more functions. Hence, a skilled person should not construe the limit of an excipient stated or illustrated in any aspect or embodiment or an example of an oral film in the present disclosure to a single function.

According to some aspects of the disclosure, the surfactant system is preferably at least one low HLB surfactant(s) (HLB from up to 12.)

According to some aspects of the disclosure, the surfactant system is preferably at least one high HLB surfactant(s) (HLB equal to 12 and above.)

According to some aspects of the disclosure, the surfactant system is preferably a combination of at least one low HLB surfactant(s) (HLB up to 12) and at least one high HLB surfactant(s) (HLB equal to 12 and above.)

The oral dissolvable film of any one of the above embodiments, further including a powder coating present on at least one external surface of the oral dissolvable film.

The oral dissolvable film of any one of the above embodiments, further including a powder coating present on two opposing external surfaces of the oral dissolvable film.

EXCIPIENTS

| Excipient | Function | Function sub-category |
|---|---|---|
| Purified Water | Solvent | |
| LABAFIL M 2125CS, Tween 20, | Co-Surfactant | Surfactant |
| Poloxamer 407 | Surfactant | |
| | Solubility enhancer | Surfactant, Amphiphilic |
| | Solubilizer | Non-volatile, Hydrophilic Character |
| Oleic acid, Recinoleic acid | Solubility enhancer | Lipophilic, Non-Amphiphilic |
| | Solubilizer | Non-volatile, Lipophilic Character |
| | Permeation enhancer | |
| L menthone, limonene, eucalyptol, | Solubilizer | Non-volatile, Lipophilic Character |
| Peppermint. | Permeation enhancer | |
| | Solubility enhancer | Lipophilic, Non-Amphiphilic |
| | Liquid flavor | |
| Benzyl Alcohol, Benzyl Acetate | Solubilizer | Non-volatile, Hydrophilic Character: |
| | | benzene derivative |
| | Permeation enhancer | |
| Methanol | Solubilizer | Volatile |
| Hydroxylated lecithin, | Co-Surfactant | Surfactant |
| | Surfactant | |
| | Solubility enhancer | Surfactant, Amphiphilic |
| | Solubilizer | Non-volatile, Hydrophilic Character |
| Cetyl pyridinium Chloride, Alpha | Permeation enhancer | |
| tocopherol, L-menthone, | | |
| Benzalkonium Chloride, Na | | |
| Glychodeoxycholate, Urea | | |
| HCL, Succinic acid, Citric acid, | Solubilizer | Non-volatile, Hydrophilic Character, pH |
| Trometamol, Ascorbic acid | | modulator |
| | Permeation enhancer | pH modulator |
| Citric acid | Plasticizer | |
| Maropitant Base emulsion | API | |
| Maropitant Base extra | | |
| Apomorphine | | |
| Buprenorphine | | |
| Dimethyltryptamine (DMT) | | |
| HPMC E4M | Suspending agent/viscosity | |
| | increasing agent | |
| PGA LV. CMC Sodium, guar gum, | Suspending agent/viscosity | |
| hydrolyzed guar gum | increasing agent | |
| | Mucoadhesive polymer | |
| CMC Sodium | Suspending agent/viscosity | |
| | increasing agent | |
| | Mucoadhesive film former. | |
| HPMC E50 | Mucoadhesive film former | Cellulosic derivative |
| | Solubilizer | Non-volatile solubilizer(s) of hydrophilic |
| | | character |
| | Solubility enhancer | Cellulosic derivative, Amphiphilic |
| | Crystal growth inhibitor | |
| PEG 300, glycerol, Propylene | Solubility enhancer | Hydrophilic, Non-Amphiphilic |
| glycol | Solubilizer | Non-volatile, Hydrophilic Character |
| | Permeation co-enhancer | |
| | Plasticizer | |
| Hydroxypropyl Cellulose (NISSO | Solubility enhancer | Cellulosic derivative, Amphiphilic |
| HPC SSL)) | | |
| Sucralose | Sweetener | |
| Magnasweet MM100 | Sweetener | |
| SavorboostTM BK | Flavor enhancer | |
| Natural Liver Powdered Flavor | Flavoring agent | |
| (PF.Inc PC-0050) | | |
| Pullulan | Mucoadhesive film former | |
| Maltodextrin (Maltrin M180) | Film forming polymer | |
| PEO WSR N80 NF | Mucoadhesive film former | |
| | Solubility enhancer | Amphiphilic |
| | Solubilizer | Non-volatile solubilizer(s) of hydrophilic |
| | | character |

### EXAMPLES

### Example 1

In this example, a solubilized pharmaceutical active phase is prepared by solubilizing a portion of the API (in our case maropitant free base) combined and mixed in solubilizing system made up of at least one of the following: fatty acid(s), benzene derivative(s), oil(s) or mixtures thereof. In parallel, an aqueous phase is prepared by mixing water, permeation co-enhancer(s), surfactant(s) and co-surfactant(s) to provide the required HLB of the solubilized pharmaceutical active phase or to promote emulsification of the later.

Next, both phases are combined (the active pharmaceutical phase and the aqueous phase) under mixing followed by the addition of a viscosity increasing agent/suspending agent to stabilize the emulsion. Once the dispersion is formed, an extra amount of the API is added to form the suspended API component and mixing is continued.

Next, a solubility enhancer(s) is added, followed by sweetener(s), Flavor enhancer(s), and flavor(s).

Finally, film forming polymer(s) are added and mixing is continued until the polymer(s) is (are) fully dissolved. Deaeration of the blend and coating it follow at a certain gap on the support liner, and at the end the coated material is placed in the oven for drying.

| 164-35 A | weight (g) | % dry | % wet |
|---|---|---|---|
| Purified Water | 17.00 | N/A | 65.53 |
| LABAFIL M 2125CS | 0.05 | 0.56 | 0.19 |
| Tween 20 | 0.48 | 5.32 | 1.84 |
| Oleic ac | 0.48 | 5.36 | 1.85 |
| Benzyl Alcohol | 0.37 | 4.17 | 1.44 |
| Maropitant | 0.39 | 4.35 | 1.50 |
| Base emulsion | | | |
| Maropitant | 1.85 | 20.68 | 7.13 |
| Base extra | | | |
| HPMC E4M | 0.17 | 1.94 | 0.67 |
| PEG 300 | 0.62 | 6.93 | 2.39 |
| Hydroxypropyl | 0.40 | 4.48 | 1.54 |
| Cellulose | | | |
| (NISSO HPC SSL)) | | | |
| Sucralose | 0.24 | 2.69 | 0.93 |
| Magnasweet MM100 | 0.24 | 2.69 | 0.93 |
| SavorboostTM BK | 0.06 | 0.67 | 0.23 |
| Natural | 0.35 | 3.92 | 1.35 |
| Liver Powdered | | | |
| Flavor (PF.Inc | | | |
| PC-0050) | | | |
| HPMC E50 | 0.24 | 2.69 | 0.93 |
| PEO WSR N80 NF | 3.00 | 33.54 | 11.56 |
| total dry | 8.94 | 100.00 | N/A |
| total wet | 25.94 | N/A | 100.00 |
| FILM TARGET WEIGHT | 239.65 mg | N/A | N/A |
| (60 mg API) | | | |

| 164-35 B | weight (g) | % dry | % wet |
|---|---|---|---|
| Purified Water | 17.00 | N/A | 65.33 |
| LABAFIL M 2125CS | 0.05 | 0.55 | 0.19 |
| Tween 20 | 0.48 | 5.28 | 1.83 |
| Oleic ac | 0.46 | 5.09 | 1.77 |
| Benzyl Alcohol | 0.36 | 3.96 | 1.37 |
| Maropitant | 0.37 | 4.13 | 1.43 |
| Base emulsion | | | |
| Maropitant | 1.88 | 20.84 | 7.22 |
| Base extra | | | |
| HPMC E4M | 0.17 | 1.92 | 0.67 |
| PEG 300 | 0.72 | 7.98 | 2.77 |
| Hydroxypropyl | 0.40 | 4.44 | 1.54 |
| Cellulose | | | |
| (NISSO HPC SSL) | | | |
| Sucralose | 0.24 | 2.66 | 0.92 |
| Magnasweet MM100 | 0.24 | 2.66 | 0.92 |
| SavorboostTM BK | 0.06 | 0.67 | 0.23 |
| Natural | 0.35 | 3.89 | 1.35 |
| Liver Powdered | | | |
| Flavor (PF.Inc | | | |
| PC-0050) | | | |
| HPMC E50 | 0.24 | 2.66 | 0.92 |
| PEO WSR N80 NF | 3.00 | 33.25 | 11.53 |
| total dry | 9.02 | 100.00 | N/A |
| total wet | 26.02 | N/A | 100.00 |
| FILM TARGET WEIGHT | 240.27 mg | N/A | N/A |
| (60 mg API) | | | |

| | weight | | |
|---|---|---|---|
| 164-28-3B | (g) | % dry | % wet |
| Purified Water | 15.10 | N/A | 61.11 |
| LABAFIL M 2125CS | 0.14 | 1.47 | 0.57 |
| Tween 20 | 0.89 | 9.37 | 3.61 |
| Oleic ac | 0.39 | 4.09 | 1.58 |
| Benzyl Alcohol | 0.39 | 4.09 | 1.58 |
| Methanol | 0.08 | N/A | 0.32 |
| Maropitant | 0.35 | 3.66 | 1.41 |
| Base emulsion | | | |
| Maropitant | 1.95 | 20.46 | 7.89 |
| Base extra | | | |
| HPMC E4M | 0.17 | 1.82 | 0.69 |
| PEG 300 | 0.55 | 5.77 | 2.23 |
| Hydroxypropyl | 0.4 | 4.20 | 1.62 |
| Cellulose | | | |
| (NISSO HPC SSL) | | | |
| Cetyl | 0.107 | 1.12 | 0.43 |
| pyridinium Chloride | | | |
| Sucralose | 0.24 | 2.52 | 0.97 |
| Magnasweet MM100 | 0.24 | 2.52 | 0.97 |
| SavorboostTM BK | 0.06 | 0.63 | 0.24 |
| Natural | 0.35 | 3.67 | 1.42 |
| Liver Powdered | | | |
| Flavor (PF.Inc | | | |
| PC-0050) | | | |
| HPMC E50 | 0.24 | 2.52 | 0.97 |
| PEO WSR N80 NF | 3.06 | 32.10 | 12.38 |
| total dry | 9.53 | 100.000 | N/A |
| total wet | 24.72 | N/A | 100.00 |
| FILM TARGET WEIGHT | 248.53 | N/A | N/A |
| (60 mg API) | | | |

| | weight | | |
|---|---|---|---|
| 164-28-3A | (g) | % dry | % wet |
| Purified Water | 15.1 | N/A | 60.97 |
| LABAFIL M 2125CS | 0.13 | 1.36 | 0.52 |
| Tween 20 | 0.89 | 9.28 | 3.59 |
| Oleic ac | 0.39 | 4.07 | 1.57 |
| Benzyl Alcohol | 0.39 | 4.07 | 1.57 |
| Methanol | 0.08 | N/A | 0.32 |
| Maropitant Base emulsion | 0.35 | 3.65 | 1.41 |
| Maropitant Base extra | 1.95 | 20.34 | 7.87 |
| HPMC E4M | 0.17 | 1.77 | 0.69 |
| Glycerol | 0.62 | 6.47 | 2.50 |
| Hydroxypropyl Cellulose | 0.4 | 4.17 | 1.62 |
| (NISSO HPC SSL) | | | |
| Cetyl pyridinium Chloride | 0.107 | 1.12 | 0.43 |
| Sucralose | 0.24 | 2.50 | 0.97 |
| Magnasweet MM100 | 0.24 | 2.50 | 0.97 |
| SavorboostTM BK | 0.06 | 0.63 | 0.24 |
| Natural Liver Powdered | 0.35 | 3.65 | 1.41 |
| Flavor (PF.Inc PC-0050) | | | |
| HPMC E50 | 0.24 | 2.50 | 0.97 |
| PEO WSR N80 NF | 3.06 | 31.92 | 12.36 |
| total dry | 9.59 | 100 | N/A |
| total wet | 24.77 | N/A | 100 |
| FILM TARGET WEIGHT (60 | 250.1 | N/A | N/A |
| mg API) | | | |

| | weight | | |
|---|---|---|---|
| 164-5E | (g) | % dry | % wet |
| Purified Water | 15.10 | N/A | 61.71 |
| LABAFIL M 2125CS | 0.06 | 0.59 | 0.23 |
| Tween 20 | 0.50 | 5.31 | 2.03 |
| Oleic acid | 1.32 | 14.06 | 5.38 |
| Maropitant Base emulsion | 0.51 | 5.44 | 2.08 |
| Maropitant Base extra | 1.79 | 19.11 | 7.32 |
| HPMC E4M | 0.17 | 1.85 | 0.71 |
| Glycerin (Glycerol USP) | 0.55 | 5.88 | 2.25 |
| Hydroxypropyl Cellulose | 0.18 | 1.92 | 0.74 |
| (NISSO HPC SSL)) | | | |
| Cetylpyridinium Chloride | 0.11 | 1.14 | 0.44 |
| Sucralose | 0.24 | 2.56 | 0.98 |
| Magnasweet MM100 | 0.24 | 2.56 | 0.98 |
| SavorboostTM BK | 0.06 | 0.64 | 0.25 |
| Natural Liver Powdered | 0.35 | 3.73 | 1.43 |
| Flavor (PF.Inc PC-0050) | | | |
| HPMC E50 | 0.24 | 2.56 | 0.98 |
| PEO WSR N80 NF | 3.06 | 32.65 | 12.50 |
| total dry | 9.37 | 100.00 | N/A |
| total wet | 24.48 | N/A | 100.00 |
| Film target weight | 244.35 mg | | |
| for 60 mg API | | | |

### Example 2

In this example, the pharmaceutical active phase include as solubilizer benzene derivative such as but not limited to benzyl alcohol, benzyl acetate, benzaldehyde.

| 164-25-1 | weight (g) | % dry | % wet |
|---|---|---|---|
| Benzyl Acetate | 1.67 | 14.33 | 5.28 |
| Maropitant | 1.20 | 10.30 | 3.79 |
| Base emulsion | | | |
| Glycerol | 0.55 | 4.72 | 1.74 |
| Purified Water | 20.00 | N/A | 63.18 |
| Labrafil M2125CS | 0.13 | 1.12 | 0.41 |
| Tween 20 | 1.18 | 10.12 | 3.73 |
| Maropitant | 1.50 | 12.87 | 4.74 |
| Base extra | | | |
| HPMC E4M | 0.30 | 2.53 | 0.93 |
| Hydroxypropyl | 0.50 | 4.29 | 1.58 |
| Cellulose | | | |
| (NISSO HPC SSL) | | | |
| Sucralose | 0.24 | 2.06 | 0.76 |
| Magnasweet MM101 | 0.24 | 2.06 | 0.76 |
| SavorboostTM BK | 0.06 | 0.51 | 0.19 |
| Natural Liver | 0.35 | 3.00 | 1.11 |
| Powdered Flavor | | | |
| (PF.Inc PC-0050) | | | |
| HPMC E50 | 0.24 | 2.06 | 0.76 |
| PEO WSR N80 NF | 3.5 | 30.03 | 11.06 |
| total dry | 11.66 | 100.00 | N/A |
| total wet | 31.66 | N/A | 100.00 |
| FILM TARGET | 259.02 mg | N/A | N/A |
| WEIGHT (60 mg API) | | | |

### Example 3

In this example, the pharmaceutical active phase includes as solubilizer an oil or mixture of oils, one or mixture saturated or unsaturated fatty acid either esterified or free fatty acid such as but not limited to alpha-Linolenic acid, alpha-Linoleic acid, arachidonic acid, palmitoleic acid, gadoleic acid, Caproic acid, heptanoic acid, 11-Dodecenoic acid, Caprylic acid, Ricinoleic acid and caproleic acid.

| 164-23-1 | weight (g) | % dry | % wet |
|---|---|---|---|
| Methanol | 0.17 | N/A | 0.53 |
| Recinoleic acid | 1.50 | 13.20 | 4.76 |
| Maropitant | 1.20 | 10.56 | 3.81 |
| Base emulsion | | | |
| Purified Water | 20.00 | N/A | 63.43 |
| Labrafil M2125CS | 0.13 | 1.14 | 0.41 |
| Tween 20 | 1.18 | 10.38 | 3.74 |
| Maropitant | 1.50 | 13.20 | 4.76 |
| Base extra | | | |
| PEG 300 | 0.55 | 4.85 | 1.75 |
| PGA LV | 0.17 | 1.52 | 0.55 |
| HPC SSL | 0.5 | 4.40 | 1.59 |
| Sucralose | 0.24 | 2.11 | 0.76 |
| Magnasweet MM101 | 0.24 | 2.11 | 0.76 |
| SavorboostTM BK | 0.06 | 0.53 | 0.19 |
| Natural Liver | 0.35 | 3.08 | 1.11 |
| Powdered Flavor | | | |
| (PF.Inc PC-0050) | | | |
| HPCMC E50 | 0.24 | 2.11 | 0.76 |
| PEO WSR N80 NF | 3.5 | 30.80 | 11.10 |
| total dry | 11.36 | 100.00 | N/A |
| total wet | 31.53 | N/A | 100.00 |
| FILM TARGET | 252.53 mg | N/A | N/A |
| WEIGHT (60 mg API) | | | |

### Example 4

In this example, the film forming matrix includes a pH modulator that help solubilizing the API. The pH modulator can be an acid (as demonstrated below in lot 164-24-2, 164-39, 164-33-3 and 164-24-1), or a basic excipient or buffer system that solubilize the whole or a part of the total strength of the API, and will provide higher surface area of the API in amorphous form that triggers permeability through the buccal mucosa.

As an example of preparation of said matrix with pH modulator: First, prepare solubilized pharmaceutical active phase by solubilizing entire API in acid condition, made by mixing water, Acidifier, plasticizer, surfactant(s) and co-surfactant(s) to provide the required HLB of the solubilized pharmaceutical active phase.

Then add viscosity increasing agent/suspending agent to stabilize the solution and prevent nucleation, add low viscosity solubilizing polymer(s), follow by sweetener(s), savor booster(s), and flavor(s).

Finally add film forming polymer(s) and keep mixing until the polymer(s) is (are) fully dissolved. Deaerate the blend then coat it at a certain gap on the support liner, and finally put the coated material in the oven for drying.

| | Theoretical | Composition | Composition |
|---|---|---|---|
| DESCRIPTION | quantity | % dry | % wet |
| lot# 164-24-2 | (g) | (w/w) | (w/w) |
| Glycerin | 0.54 | 7.87 | 2.49 |
| (Glycerol USP) | | | |
| Purified Water | 14.79 | N/A | 68.30 |
| HCl | 0.20 | 2.84 | 0.90 |
| Labrafil 2125CS | 0.11 | 1.53 | 0.48 |
| Tween 20 | 0.48 | 6.99 | 2.22 |
| Maropitant Base | 1.70 | 24.77 | 7.85 |
| Hydroxypropyl | 0.17 | 2.51 | 0.79 |
| Cellulose | | | |
| (NISSO HPC SSL)) | | | |
| Sucralose | 0.23 | 3.38 | 1.07 |
| Ammonium | 0.23 | 3.38 | 1.07 |
| Glycyrrhizinate | | | |
| (Magnasweet MM100) | | | |
| Yeast Extract | 0.06 | 0.83 | 0.26 |
| (Ajinomoto Brand | | | |
| Savorboost ™ BK) | | | |
| Natural Liver | 0.34 | 4.95 | 1.57 |
| Powdered Flavor | | | |
| (PF.Inc PC-0050) | | | |
| Hypromellose | 0.20 | 2.91 | 0.92 |
| 2910 USP (HPMC E50) | | | |
| Polyethylene | 2.00 | 29.14 | 9.24 |
| Oxide (Polyox Sentry | | | |
| WS RN80 NF) | | | |
| Hydrolyzed | 0.50 | 7.29 | 2.31 |
| Guar Gum | | | |
| HPMC E4M | 0.11 | 1.60 | 0.51 |
| TOTAL WET | 21.65 | N/A | 100.00 |
| TOTAL DRY | 6.86 | 100.00 | |
| | Target | 242.22 mg | |
| | weight for | | |
| | 60 mg API | | |

| | Theoretical | Composition | Composition |
|---|---|---|---|
| DESCRIPTION | quantity | % dry | % wet |
| lot# 164-39 | (g) | (w/w) | (w/w) |
| Glycerin | 4.48 | 7.83 | 2.49 |
| (Glycerol USP) | | | |
| Purified Water | 122.78 | N/A | 68.21 |
| HCl | 1.62 | 2.83 | 0.90 |
| Labrafil M2125CS | 0.86 | 1.51 | 0.48 |
| Tween 20 | 3.98 | 6.95 | 2.21 |
| Maropitant Base | 14.09 | 24.63 | 7.83 |
| Hydroxypropyl | 0.99 | 1.73 | 0.55 |
| Methylcellulose | | | |
| (Metolose 90SH-4000SR) | | | |
| Hydroxypropyl | 1.66 | 2.89 | 0.92 |
| Cellulose | | | |
| (NISSO HPC SSL)) | | | |
| Sucralose | 1.93 | 3.37 | 1.07 |
| Ammonium | 1.93 | 3.37 | 1.07 |
| Glycyrrhizinate(Magnasweet | | | |
| MM100) | | | |
| Yeast Extract | 0.47 | 0.82 | 0.26 |
| (Ajinomoto Brand | | | |
| SavorboostTM BK) | | | |
| Natural Liver | 2.83 | 4.94 | 1.57 |
| Powdered Flavor (PF.Inc | | | |
| PC-0050) | | | |
| Hypromellose | 1.66 | 2.89 | 0.92 |
| 2910 USP (HPMC E50) | | | |
| Polyethylene Oxide | 20.74 | 36.24 | 11.52 |
| (Polyox Sentry WSR | | | |
| N80 NF) | | | |
| TOTAL WET | 180.000 | N/A | 100.00 |
| TOTAL DRY | 57.222 | 100.00 | |
| | Target | 243.60 mg | |
| | weight | | |
| | for (60 mg | | |
| | API) | | |

| | Theoretical | Composition | Composition |
|---|---|---|---|
| DESCRIPTION | quantity | % dry | % wet |
| lot# 164-33-3 | (g) | (w/w) | (w/w) |
| Glycerin | 0.540 | 7.04 | 2.38 |
| (Glycerol USP) | | | |
| Purified Water | 15.000 | N/A | 66.15 |
| Succinic acid | 0.800 | 10.42 | 3.53 |
| Labrafil M2125CS | 0.117 | 1.52 | 0.52 |
| Tween 20 | 0.513 | 6.68 | 2.26 |
| Maropitant Base | 1.800 | 23.45 | 7.94 |
| HPMC E4M | 0.171 | 2.23 | 0.75 |
| Hydroxypropyl | 0.172 | 2.24 | 0.76 |
| Cellulose | | | |
| (NISSO HPC SSL)) | | | |
| Sucralose | 0.232 | 3.02 | 1.02 |
| Ammonium | 0.232 | 3.02 | 1.02 |
| Glycyrrhizinate | | | |
| (Magnasweet MM100) | | | |
| Yeast Extract | 0.057 | 0.75 | 0.25 |
| (Ajinomoto Brand | | | |
| Savorboost ™ BK) | | | |
| Natural Liver | 0.340 | 4.43 | 1.50 |
| Powdered Flavor | | | |
| (PF.Inc PC-0050) | | | |
| Hypromellose | 0.200 | 2.61 | 0.88 |
| 2910 USP (HPMC E50) | | | |
| Polyethylene Oxide | 2.000 | 26.06 | 8.82 |
| (Polyox Sentry WSR N80 NF) | | | |
| Hydrolyzed med | 0.500 | 6.51 | 2.21 |
| viscosity guar gum | | | |
| Total | 22.675 | 100.00 | 100.00 |
| | Target | 255.82 mg | |
| | weight for | | |
| | (60 mg API) | | |

| | Theoretical | Composition | Composition |
|---|---|---|---|
| DESCRIPTION | quantity | % dry | % wet |
| lot# 164-24-1 | (g) | (w/w) | (w/w) |
| Glycerin | 0.540 | 7.13 | 2.39 |
| (Glycerol USP) | | | |
| Purified Water | 15.000 | N/A | 66.46 |
| Succinic acid | 0.800 | 10.57 | 3.54 |
| Labrafil M2125CS | 0.105 | 1.39 | 0.47 |
| Tween 20 | 0.480 | 6.34 | 2.13 |
| Maropitant Base | 1.800 | 23.78 | 7.98 |
| HPMC E4M | 0.110 | 1.45 | 0.49 |
| Hydroxypropyl | 0.172 | 2.27 | 0.76 |
| Cellulose | | | |
| (NISSO HPC SSL)) | | | |
| Sucralose | 0.232 | 3.07 | 1.03 |
| Ammonium | 0.232 | 3.07 | 1.03 |
| Glycyrrhizinate | | | |
| (Magnasweet MM100) | | | |
| Yeast Extract | 0.057 | 0.76 | 0.25 |
| (Ajinomoto Brand | | | |
| Savorboost ™ BK) | | | |
| Natural Liver | 0.340 | 4.50 | 1.51 |
| Powdered Flavor | | | |
| (PF.Inc PC-0050) | | | |
| Hypromellose | 0.200 | 2.64 | 0.89 |
| 2910 USP (HPMC E50) | | | |
| Polyethylene Oxide | 2.500 | 33.03 | 11.08 |
| (Polyox Sentry WSR | | | |
| N80 NF) | | | |
| Total | 22.569 | 100.00 | 100.00 |
| | Target | 252.29 mg | |
| | weight for | | |
| | (60 mg API) | | |

### Example 5

Film forming matrix combining API solubilized with pH modulator and solubilizing system made of one of the following: fatty acid(s), benzene derivative(s), oil(s) or mixture of the above. Said matrix will have API fully solubilized.

| | Theoretical | Composition | Composition |
|---|---|---|---|
| DESCRIPTION | quantity | % dry | % wet |
| lot# 164-33-M | (g) | (w/w) | (w/w) |
| Glycerin | 0.54 | 6.45 | 2.31 |
| (Glycerol USP) | | | |
| Purified Water | 15 | N/A | 64.17 |
| Succinic acid | 0.8 | 9.55 | 3.42 |
| Oleic acid | 0.9 | 10.75 | 3.85 |
| Labrafil M2125CS | 0.117 | 1.40 | 0.50 |
| Tween 20 | 0.513 | 6.13 | 2.19 |
| Maropitant Base | 2.1 | 25.08 | 8.98 |
| HPMC E4M | 0.171 | 2.04 | 0.73 |
| Hydroxypropyl | 0.172 | 2.05 | 0.74 |
| Cellulose | | | |
| (NISSO HPC SSL)) | | | |
| Sucralose | 0.232 | 2.77 | 0.99 |
| Ammonium | 0.232 | 2.77 | 0.99 |
| Glycyrrhizinate | | | |
| (Magnasweet MM100) | | | |
| Yeast Extract | 0.057 | 0.68 | 0.24 |
| (Ajinomoto Brand | | | |
| SavorboostTM BK) | | | |
| Natural Liver | 0.34 | 4.06 | 1.45 |
| Powdered Flavor | | | |
| (PF.Inc PC-0050) | | | |
| Hypromellose | 0.2 | 2.39 | 0.86 |
| 2910 USP (HPMC E50) | | | |
| Polyethylene Oxide | 2 | 23.88 | 8.56 |
| (Polyox Sentry WSR | | | |
| N80 NF) | | | |
| Total | 23.37 | 100 | 100 |
| | Target | 239.26 mg | |
| | weight for | | |
| | (60 mg API) | | |

### Example 6

In this example, the active pharmaceutical phase uses longer chain fatty acid or higher amount of lipid based excipients or benzene derivatives in the matrix and cause performance issues (longer disintegration time, low permeation, oiliness on surface.)
1—Said pharmaceutical active phase include two types of solubilizer one an organic solvent and the second a oil or mixture of oils, one or mixture saturated or unsaturated fatty acid either esterified or free fatty acid such as but not limited to alpha-Linolenic acid, alpha-Linoleic acid, arachidonic acid, palmitoleic acid, gadoleic acid, Caproic acid, heptanoic acid, 11-Dodecenoic acid, Caprylic acid, Ricinoleic acid and caproleic acid.
2—The film matrix do not have permeation co-enhancer or having the non functional one.
3—Pharmaceutical active phase where the majority of the solubilizer(s) is (are) essential oils such as L menthone, limonene, eucalyptol, Peppermint.
4—Pharmaceutical active phase use higher ratio of lipid based excipient.
5—Film matrix where the fatty acid(s) or its (their) derivative(s) is (are) not less than 10% and surfactant(s) is (are) not less than 10%.
6—Film matrix where the fatty acid(s) is (are) not less than 14%.
7—Pharmaceutical active phase use at least organic solvent(s) to solubilize the API.

### Example 7

In this example, Film forming matrix of lot 164-5 A made in neutral condition where a portion of the API is solubilized in oleic acid and emulsified with Labrafil and Tween, and the rest of the API (extra) is suspended. The glycerol was used as permeation co-enhancer for the permeation and as plasticizer for the film.

| Ingredients | weight | | |
|---|---|---|---|
| list 164-5 A | (g) | % dry | % wet |
| Purified Water | 15.10 | N/A | 61.98 |
| LABAFIL M 2125CS | 0.06 | 0.60 | 0.23 |
| Tween 20 | 0.50 | 5.37 | 2.04 |
| Oleic ac | 1.32 | 14.22 | 5.41 |
| Maropitant Base emulsion | 0.51 | 5.50 | 2.09 |
| Maropitant Base extra | 1.79 | 19.34 | 7.35 |
| HPMC E4M | 0.17 | 1.87 | 0.71 |
| Glycerin (Glycerol USP) | 0.55 | 5.94 | 2.26 |
| Hydroxypropyl Cellulose | 0.18 | 1.94 | 0.74 |
| (NISSO HPC SSL)) | | | |
| Sucralose | 0.24 | 2.59 | 0.98 |
| Magnasweet MM100 | 0.24 | 2.59 | 0.98 |
| SavorboostTM BK | 0.06 | 0.65 | 0.25 |
| Natural Liver Powdered | 0.35 | 3.78 | 1.44 |
| Flavor (PF.Inc PC-0050) | | | |
| HPMC E50 | 0.24 | 2.59 | 0.98 |
| PEO WSR N80 NF | 3.06 | 33.02 | 12.56 |
| total dry | 9.27 | 100.00 | N/A |
| total wet | 24.37 | N/A | 100.00 |
| FILM TARGET WEIGHT | 241.56 MG | | |
| (60 mg API) | | | |

### Example 8

In this example, the film forming matrix of lot 164-5 B made in pH around 8 to increase to uncharged part of the API which is favourable to permeate across the buccal mucosa. A portion of the API is solubilized in oleic acid and emulsified with Labrafil and Tween, and the rest of the API (extra) is suspended. The glycerol was used as permeation co-enhancer for the permeation and as plasticizer for the film.

| Ingredients | weight | | |
|---|---|---|---|
| list 164-5 B | (g) | % dry | % wet |
| Purified Water | 15.10 | N/A | 61.04 |
| LABAFIL M 2125CS | 0.06 | 0.58 | 0.23 |
| Tween 20 | 0.50 | 5.16 | 2.01 |
| Oleic ac | 1.32 | 13.67 | 5.33 |
| Maropitant Base emulsion | 0.51 | 5.29 | 2.06 |
| Maropitant Base extra | 1.79 | 18.59 | 7.24 |
| HPMC E4M | 0.17 | 1.80 | 0.70 |
| Glycerin (Glycerol USP) | 0.55 | 5.71 | 2.23 |
| Hydroxypropyl Cellulose | 0.18 | 1.87 | 0.73 |
| (NISSO HPC SSL)) | | | |
| Trometamol | 0.372 | 3.86 | 1.50 |
| Sucralose | 0.24 | 2.49 | 0.97 |
| Magnasweet MM100 | 0.24 | 2.49 | 0.97 |
| SavorboostTM BK | 0.06 | 0.62 | 0.24 |
| Natural Liver Powdered | 0.35 | 3.63 | 1.41 |
| Flavor (PF.Inc PC-0050) | | | |
| HPMC E50 | 0.24 | 2.49 | 0.97 |
| PEO WSR N80 NF | 3.06 | 31.75 | 12.37 |
| total dry | 9.64 | 100.00 | N/A |
| total wet | 24.74 | N/A | 100.00 |
| FILM TARGET WEIGHT | 251.26 mg | | |
| (60 mg API) | | | |

### Example 9

In this example, Film forming matrix of lot 164-5 C, lot 164-5 D, lot 164-5 B are made in neutral condition where a portion of the API is solubilized in oleic acid and emulsified with Labrafil and Tween, and the rest of the API (extra) is suspended. The glycerol was used as permeation co-enhancer for the permeation and as plasticizer for the film. Permeation enhancer where added in each prototypes.

| | 164-5 C | | | 164-5 D | | | 164-5 E | | |
|---|---|---|---|---|---|---|---|---|---|
| Ingredients | weight | | | weight | | | weight | | |
| list | (g) | % dry | % wet | (g) | % dry | % wet | (g) | % dry | % wet |
| Purified | 15.10 | N/A | 61.67 | 15.10 | N/A | 61.47 | 15.10 | N/A | 61.71 |
| Water | | | | | | | | | |
| LABAFIL M | 0.06 | 0.59 | 0.23 | 0.06 | 0.59 | 0.23 | 0.06 | 0.59 | 0.23 |
| 2125CS | | | | | | | | | |
| Tween 20 | 0.50 | 5.30 | 2.03 | 0.50 | 5.26 | 2.03 | 0.50 | 5.31 | 2.03 |
| Oleic ac | 1.32 | 14.03 | 5.38 | 1.32 | 13.92 | 5.36 | 1.32 | 14.06 | 5.38 |
| Maropitant | 0.51 | 5.43 | 2.08 | 0.51 | 5.39 | 2.08 | 0.51 | 5.44 | 2.08 |
| Base | | | | | | | | | |
| emulsion | | | | | | | | | |
| Maropitant | 1.79 | 19.08 | 7.32 | 1.79 | 18.92 | 7.29 | 1.79 | 19.11 | 7.32 |
| Base extra | | | | | | | | | |
| HPMC E4M | 0.17 | 1.84 | 0.71 | 0.17 | 1.83 | 0.70 | 0.17 | 1.85 | 0.71 |
| Glycerin | 0.55 | 5.87 | 2.25 | 0.55 | 5.82 | 2.24 | 0.55 | 5.88 | 2.25 |
| (Glycerol | | | | | | | | | |
| USP) | | | | | | | | | |
| Hydroxypropyl | 0.18 | 1.92 | 0.73 | 0.18 | 1.90 | 0.73 | 0.18 | 1.92 | 0.74 |
| Cellulose | | | | | | | | | |
| (NISSO | | | | | | | | | |
| HPC SSL)) | | | | | | | | | |
| * | 0.122 | 1.30 | 0.50 | 0.201 | 2.12 | 0.82 | 0.107 | 1.14 | 0.44 |
| Sucralose | 0.24 | 2.56 | 0.98 | 0.24 | 2.54 | 0.98 | 0.24 | 2.56 | 0.98 |
| Magnasweet | 0.24 | 2.56 | 0.98 | 0.24 | 2.54 | 0.98 | 0.24 | 2.56 | 0.98 |
| MM100 | | | | | | | | | |
| SavorboostTM | 0.06 | 0.64 | 0.24 | 0.06 | 0.63 | 0.24 | 0.06 | 0.64 | 0.25 |
| BK | | | | | | | | | |
| Natural Liver | 0.35 | 3.73 | 1.43 | 0.35 | 3.70 | 1.42 | 0.35 | 3.73 | 1.43 |
| Powdered | | | | | | | | | |
| Flavor | | | | | | | | | |
| (PF. Inc PC- | | | | | | | | | |
| 0050) | | | | | | | | | |
| HPMC E50 | 0.24 | 2.56 | 0.98 | 0.24 | 2.54 | 0.98 | 0.24 | 2.56 | 0.98 |
| PEO WSR | 3.06 | 32.59 | 12.49 | 3.06 | 32.32 | 12.45 | 3.06 | 32.65 | 12.50 |
| N80 NF | | | | | | | | | |
| total dry | 9.39 | 100.00 | N/A | 9.47 | 100.00 | N/A | 9.37 | 100.00 | N/A |
| total wet | 24.49 | N/A | 100.00 | 24.57 | N/A | 100.00 | 24.48 | N/A | 100.00 |
| FILM | 244.74 | | | 246.80 | | | 244.35 | | |
| TARGET | | | | | | | | | |
| WEIGHT | | | | | | | | | |
| (60 mg API) | | | | | | | | | |

| | | |
|---|---|---|
| * Permeation enhancer: | * Permeation enhancer: | * Permeation enhancer: |
| L-menthone | Alpha tocopherol | Cetylpyridinium Chloride |

### Example 10

In this example, the film forming matrix of lot 164-6 A is made in neutral condition while lot 164-6 B is made in pH of around 8 to increase to uncharged part of the API which is favourable to permeate cross the buccal mucosa. Lot 164-6 C is made in neutral condition and a permeation enhancer(s) where added. For the three above-mentioned films, a portion of the API is solubilized in oleic acid and emulsified with Labrafil and poloxamer, and the rest of the API (extra) is suspended. The glycerol was used as permeation co-enhancer for the permeation and as plasticizer for the film.

| | 164-6 A | | | 164-6 B | | | 164-6 C | | |
|---|---|---|---|---|---|---|---|---|---|
| | weight | | | weight | | | weight | | |
| Ingredients list | (g) | % dry | % wet | (g) | % dry | % wet | (g) | % dry | % wet |
| Purified Water | 15.10 | N/A | 61.98 | 15.10 | N/A | 61.04 | 15.10 | N/A | 61.71 |
| LABAFIL M | 0.26 | 2.77 | 1.05 | 0.06 | 0.58 | 0.23 | 0.06 | 0.59 | 0.23 |
| 2125CS | | | | | | | | | |
| Poloxamer | 0.30 | 3.20 | 1.22 | 0.50 | 5.16 | 2.01 | 0.50 | 5.31 | 2.03 |
| 407 | | | | | | | | | |
| Oleic ac | 1.32 | 14.22 | 5.41 | 1.32 | 13.67 | 5.33 | 1.32 | 14.06 | 5.38 |
| Maropitant | 0.51 | 5.50 | 2.09 | 0.51 | 5.29 | 2.06 | 0.51 | 5.44 | 2.08 |
| Base | | | | | | | | | |
| emulsion | | | | | | | | | |
| Maropitant | 1.79 | 19.34 | 7.35 | 1.79 | 18.59 | 7.24 | 1.79 | 19.11 | 7.32 |
| Base extra | | | | | | | | | |
| HPMC E4M | 0.17 | 1.87 | 0.71 | 0.17 | 1.80 | 0.70 | 0.17 | 1.85 | 0.71 |
| Glycerin | 0.55 | 5.94 | 2.26 | 0.55 | 5.71 | 2.23 | 0.55 | 5.88 | 2.25 |
| (Glycerol | | | | | | | | | |
| USP) | | | | | | | | | |
| Hydroxypropyl | 0.18 | 1.94 | 0.74 | 0.18 | 1.87 | 0.73 | 0.18 | 1.92 | 0.74 |
| Cellulose | | | | | | | | | |
| (NISSO HPC | | | | | | | | | |
| SSL)) | | | | | | | | | |
| * | 0 | 0.00 | 0.00 | 0.372 | 3.86 | 1.50 | 0.107 | 1.14 | 0.44 |
| Sucralose | 0.24 | 2.59 | 0.98 | 0.24 | 2.49 | 0.97 | 0.24 | 2.56 | 0.98 |
| Magnasweet | 0.24 | 2.59 | 0.98 | 0.24 | 2.49 | 0.97 | 0.24 | 2.56 | 0.98 |
| MM100 | | | | | | | | | |
| SavorboostTM | 0.06 | 0.65 | 0.25 | 0.06 | 0.62 | 0.24 | 0.06 | 0.64 | 0.25 |
| BK | | | | | | | | | |
| Natural Liver | 0.35 | 3.78 | 1.44 | 0.35 | 3.63 | 1.41 | 0.35 | 3.73 | 1.43 |
| Powdered | | | | | | | | | |
| Flavor (PF. Inc | | | | | | | | | |
| PC-0050) | | | | | | | | | |
| HPMC E50 | 0.24 | 2.59 | 0.98 | 0.24 | 2.49 | 0.97 | 0.24 | 2.56 | 0.98 |
| PEO WSR | 3.06 | 33.02 | 12.56 | 3.06 | 31.75 | 12.37 | 3.06 | 32.65 | 12.50 |
| N80 NF | | | | | | | | | |
| total dry | 9.27 | 100.00 | N/A | 9.64 | 100.00 | N/A | 9.37 | 100.00 | N/A |
| total wet | 24.37 | N/A | 100.00 | 24.74 | N/A | 100.00 | 24.48 | N/A | 100.00 |
| FILM | 241.56 mg | | | 251.26 mg | | | 244.35 mg | | |
| TARGET | | | | | | | | | |
| WEIGHT (60 | | | | | | | | | |
| mg API) | | | | | | | | | |

| | | |
|---|---|---|
| * none | * pH adjustment with Tris | * Permeation enhancer: |

| | |
|---|---|
| (OH methyl) aminomethan | Benzalkonium Chloride |

### Example 11

In this example, the film forming matrix of lot 164-6 D, and 164-6 E are made in neutral condition and a permeation enhancer where added in each prototype. For both lots, a portion of the API is solubilized in oleic acid and emulsified with Labrafil and poloxamer, and the rest of the API (extra) is suspended. The glycerol was used as permeation co-enhancer for the permeation and as plasticizer for the film.

| | 164-6 D | | | 164-6 E | | |
|---|---|---|---|---|---|---|
| Ingredients list | weight (g) | % dry | % wet | weight (g) | % dry | % wet |
| Purified Water | 15.10 | N/A | 61.71 | 15.10 | N/A | 61.42 |
| LABAFIL M | 0.06 | 0.59 | 0.23 | 0.06 | 0.59 | 0.23 |
| 2125CS | | | | | | |
| Poloxamer 407 | 0.50 | 5.31 | 2.03 | 0.50 | 5.25 | 2.02 |
| Oleic ac | 1.32 | 14.06 | 5.38 | 1.32 | 13.89 | 5.36 |
| Maropitant Base | 0.51 | 5.44 | 2.08 | 0.51 | 5.37 | 2.07 |
| emulsion | | | | | | |
| Maropitant Base | 1.79 | 19.11 | 7.32 | 1.79 | 18.88 | 7.29 |
| extra | | | | | | |
| HPMC E4M | 0.17 | 1.85 | 0.71 | 0.17 | 1.82 | 0.70 |
| Glycerin (Glycerol | 0.55 | 5.88 | 2.25 | 0.55 | 5.80 | 2.24 |
| USP) | | | | | | |
| Hydroxypropyl | 0.18 | 1.92 | 0.74 | 0.18 | 1.90 | 0.73 |
| Cellulose (NISSO | | | | | | |
| HPC SSL)) | | | | | | |
| * | 0.107 | 1.14 | 0.44 | 0.222 | 2.34 | 0.90 |
| Sucralose | 0.24 | 2.56 | 0.98 | 0.24 | 2.53 | 0.98 |
| Magnasweet | 0.24 | 2.56 | 0.98 | 0.24 | 2.53 | 0.98 |
| MM100 | | | | | | |
| SavorboostTM BK | 0.06 | 0.64 | 0.25 | 0.06 | 0.63 | 0.24 |
| Natural Liver | 0.35 | 3.73 | 1.43 | 0.35 | 3.69 | 1.42 |
| Powdered Flavor | | | | | | |
| (PF. Inc PC-0050) | | | | | | |
| HPMC E50 | 0.24 | 2.56 | 0.98 | 0.24 | 2.53 | 0.98 |
| PEO WSR N80 | 3.06 | 32.65 | 12.50 | 3.06 | 32.25 | 12.44 |
| NF | | | | | | |
| total dry | 9.37 | 100.00 | N/A | 9.49 | 100.00 | N/A |
| total wet | 24.48 | N/A | 100.00 | 24.59 | N/A | 100.00 |
| Film target weight | 244.35 mg | | | 247.35 mg | | |
| (60 mg API) | | | | | | |

| N/A | * Permeation enhancer: | * Permeation enhancer: Urea |
|---|---|---|
| | Na Glychodeoxycholate | |

### Example 12

In this example, Film forming matrix of lot 164-13 made in neutral condition where the API is suspended. The main film forming polymer is pullulan.

| | Theoretical | Composition | Composition |
|---|---|---|---|
| lot# 164-13 | quantity | % dry | % wet |
| Ingredients | (g) | (w/w) | (w/w) |
| Glycerin | 0.800 | 11.45 | 3.64 |
| (Glycerol USP) | | | |
| Purified Water | 15.000 | — | 68.23 |
| Labrafil M2125CS | 0.600 | 8.59 | 2.73 |
| Maropitant Base | 1.770 | 25.34 | 8.05 |
| Polygal | 0.172 | 2.47 | 0.78 |
| guar hydrolyzed | | | |
| sucralose | 0.232 | 3.32 | 1.06 |
| Magnasweet MM100 | 0.232 | 3.32 | 1.06 |
| Yeast | 0.057 | 0.82 | 0.26 |
| Extract | | | |
| (Ajinomoto Brand | | | |
| Savorboost ™ BK) | | | |
| Natural | 0.340 | 4.87 | 1.55 |
| Liver Powdered | | | |
| Flavor (PF.Inc | | | |
| PC-0050) | | | |
| Maltodextrin | 0.590 | 8.45 | 2.68 |
| (Maltrin M180) | | | |
| Pullulan | 2.190 | 31.36 | 9.96 |
| total | 21.984 | 100.00 | 100.00 |
| | Target weight | 236.75 mg | |
| | for (60 mg | | |
| | API) | | |

### Example 13

In this example, Film forming matrix of lot 164-15-1, and 164-15-2 are made in neutral condition and where benzyl alcohol was used to solubilize a portion of the API and emulsified with tween only (lot 164-15-1) or Labrafil and tween (lot 164-15-2). The rest of the API (extra) is suspended. The glycerol was used as permeation co-enhancer for the permeation and as plasticizer for the film.

| | 164-15-1 | | | | 164-15-2 | | |
|---|---|---|---|---|---|---|---|
| | weight | | | | weight | | |
| Ingredients | (g) | % dry | % wet | Ingredients | (g) | % dry | % wet |
| Purified Water | 20.00 | N/A | 63.22 | Purified Water | 20.00 | N/A | 63.22 |
| N/A | | | | Labrafil | 0.10 | 0.82 | 0.30 |
| | | | | M2125CS | | | |
| Tween 20 | 1.00 | 8.60 | 3.16 | Tween 20 | 0.91 | 7.78 | 2.86 |
| Benzyl Alcohol | 2.42 | 20.80 | 7.65 | Benzyl Alcohol | 2.42 | 20.80 | 7.65 |
| Maropitant Base | 1.00 | 8.60 | 3.16 | Maropitant Base | 1.00 | 8.60 | 3.16 |
| emulsion | | | | emulsion | | | |
| Maropitant Base | 1.80 | 15.47 | 5.69 | Maropitant Base | 1.80 | 15.47 | 5.69 |
| extra | | | | extra | | | |
| Glycerin | 0.55 | 4.73 | 1.74 | Glycerin | 0.55 | 4.73 | 1.74 |
| (Glycerol USP) | | | | (Glycerol USP) | | | |
| HPMC E4M | 0.17 | 1.49 | 0.55 | HPMC E4M | 0.17 | 1.49 | 0.55 |
| HPC SSL | 0.5 | 4.30 | 1.58 | HPC SSL | 0.5 | 4.30 | 1.58 |
| Sucralose | 0.24 | 2.06 | 0.76 | Sucralose | 0.24 | 2.06 | 0.76 |
| Magnasweet | 0.24 | 2.06 | 0.76 | Magnasweet | 0.24 | 2.06 | 0.76 |
| MM100 | | | | MM100 | | | |
| SavorboostTM | 0.06 | 0.52 | 0.19 | SavorboostTM | 0.06 | 0.52 | 0.19 |
| BK | | | | BK | | | |
| Natural Liver | 0.35 | 3.01 | 1.11 | Natural Liver | 0.35 | 3.01 | 1.11 |
| Powdered | | | | Powdered | | | |
| Flavor (PF. Inc | | | | Flavor (PF. Inc | | | |
| PC-0050) | | | | PC-0050) | | | |
| HPMC E50 | 0.24 | 2.06 | 0.76 | HPMC E50 | 0.24 | 2.06 | 0.76 |
| PEO WSR N80 | 3.06 | 26.30 | 9.67 | PEO WSR N80 | 3.06 | 26.30 | 9.67 |
| NF | | | | NF | | | |
| total dry | 11.63 | 100.00 | N/A | total dry | 11.63 | 100.00 | N/A |
| total wet | 31.63 | N/A | 100.00 | total wet | 31.63 | N/A | 100.00 |
| Film target | 249.29 mg | | | Film target | 249.29 mg | | |
| weight (60 mg | | | | weight (60 mg | | | |
| API) | | | | API) | | | |

### Example 14

In this example, the film forming matrix of lot 164-16 made in neutral conditions where a portion of the API is solubilized in oleic acid and emulsified with Labrafil and Tween, and the rest of the API (extra) is suspended. The glycerol was used as permeation co-enhancer for the permeation and as plasticizer for the film. The main film forming polymer are poly acid (CMC.)

| lot 164-16 - | Amount | | |
|---|---|---|---|
| Ingredients | (g) | % Wet | % Dry |
| Purified water (USP) | 18.00 | 64.29 | N/A |
| oleic ac | 1.42 | 5.08 | 14.22 |
| Glycerin (Glycerol | 0.59 | 2.12 | 5.94 |
| USP) | | | |
| Labrafil M2125CS | 0.06 | 0.21 | 0.60 |
| Tween 20 | 0.54 | 1.92 | 5.37 |
| API emulsion | 0.56 | 1.99 | 5.58 |
| HPMC E4M | 0.18 | 0.64 | 1.80 |
| API extra | 1.93 | 6.91 | 19.34 |
| HPC SSL | 0.19 | 0.69 | 1.94 |
| Sucralose | 0.26 | 0.93 | 2.60 |
| Magnasweet MM100 | 0.26 | 0.93 | 2.60 |
| Savorboost BK | 0.07 | 0.23 | 0.65 |
| Flavor PF PC-0050 | 0.37 | 1.32 | 3.70 |
| HPMC E50 | 0.26 | 0.94 | 2.64 |
| CMC Sodium (Cekol | 1.30 | 4.63 | 12.97 |
| 150) | | | |
| CMC Sodium (Cekol | 2.01 | 7.16 | 20.05 |
| 30) | | | |
| Total Wet | 28.00 | 100.00 | N/A |
| Total Dry | 10.00 | N/A | 100.00 |
| % Dry | 35.71 | N/A | N/A |
| Film target weight | 240.80 mg | N/A | N/A |
| (60 mg API) | | | |

### Example 15

In this example, the film forming matrix of lot 164-28-6A and lot 164-28-6A are made in neutral condition where a portion of the API is solubilized in oleic acid and emulsified with Labrafil and Tween, the rest of the API (extra) is suspended. The total oleic acid is less than 5% dry, glycerol and propylene glycol were used as permeation co-enhancers for the permeation and as plasticizers for the film.

| 164-28-6A | weight (g) | % dry | % wet | 164-28-6B | weight (g) | % dry | % wet |
|---|---|---|---|---|---|---|---|
| Purified | 17.00 | N/A | 66.46 | Purified | 17.00 | N/A | 66.46 |
| Water | | | | Water | | | |
| LABAFIL | 0.06 | 0.70 | 0.23 | LABAFIL | 0.07 | 0.76 | 0.25 |
| 2125 | | | | 2125 | | | |
| Tween 20 | 0.50 | 5.83 | 1.95 | Tween 20 | 0.50 | 5.83 | 1.95 |
| Oleic ac | 0.40 | 4.66 | 1.56 | Oleic ac | 0.40 | 4.66 | 1.56 |
| Maropitant | 0.20 | 2.38 | 0.80 | Maropitant | 0.20 | 2.38 | 0.80 |
| Base | | | | Base | | | |
| emulsion | | | | emulsion | | | |
| Maropitant | 2.10 | 24.48 | 8.21 | Maropitant | 2.10 | 24.43 | 8.19 |
| Base extra | | | | Base extra | | | |
| HPMC E4M | 0.17 | 2.02 | 0.68 | HPMC E4M | 0.17 | 2.02 | 0.68 |
| Glycerin | 0.55 | 6.42 | 2.15 | Propylen | 0.55 | 6.42 | 2.15 |
| (Glycerol | | | | Glycol | | | |
| USP) | | | | | | | |
| Hydroxy- | 0.4 | 4.66 | 1.56 | Hydroxy- | 0.4 | 4.66 | 1.56 |
| propyl | | | | propyl | | | |
| Cellulose | | | | Cellulose | | | |
| (NISSO HPC | | | | (NISSO HPC | | | |
| SSL)) | | | | SSL)) | | | |
| Sucralose | 0.24 | 2.80 | 0.94 | Sucralose | 0.24 | 2.80 | 0.94 |
| Magnasweet | 0.24 | 2.80 | 0.94 | Magnasweet | 0.24 | 2.80 | 0.94 |
| MM100 | | | | MM100 | | | |
| SavorboostTM | 0.06 | 0.70 | 0.23 | SavorboostTM | 0.06 | 0.70 | 0.23 |
| BK | | | | BK | | | |
| Natural Liver | 0.35 | 4.08 | 1.37 | Natural Liver | 0.35 | 4.08 | 1.37 |
| Powdered | | | | Powdered | | | |
| Flavor | | | | Flavor | | | |
| (PF. Inc PC- | | | | (PF. Inc PC- | | | |
| 0050) | | | | 0050) | | | |
| HPMC E50 | 0.24 | 2.80 | 0.94 | HPMC E50 | 0.24 | 2.80 | 0.94 |
| PEO WSR | 3.06 | 35.67 | 11.96 | PEO WSR | 3.06 | 35.67 | 11.96 |
| N80 NF | | | | N80 NF | | | |
| total dry | 8.58 | 100.00 | | total dry | 8.58 | 100.00 | N/A |
| total wet | 25.58 | N/A | 100.00 | total wet | 25.58 | N/A | 100.00 |
| Film Target | 223.37 mg | N/A | N/A | Film Target | 223.79 mg | N/A | N/A |
| weight (60 | | | | weight (60 | | | |
| mg API) | | | | mg API) | | | |

### Example 16

In the following example, film matrix of lot 164-Ta made in neutral condition where a portion of poorly soluble Tadalafil is solubilized in propylene glycol Monocaprylate, Benzyl Alcohol, and PEG 300 and then emulsified with labrafil M2125CS and Tween 20, the rest of the API is suspended. The PEG 300 was used as solubilizer, permeation co-enhancer and as plasticizer for the film.

| DESCRIPTION | | | |
|---|---|---|---|
| lot# 164-Ta | weight (g) | % dry | % wet |
| Purified Water | 15.1 | N/A | 62.63 |
| LABAFIL M 2125CS | 0.14 | 1.554 | 0.58 |
| Tween 20 | 0.89 | 9.878 | 3.69 |
| Propylene | 0.39 | 4.329 | 1.62 |
| Glycol | | | |
| Monocaprylate | | | |
| Benzyl Alcohol | 0.39 | 4.329 | 1.62 |
| Tadalafil emulsion | 0.35 | 3.885 | 1.45 |
| Tadalafil extra | 1.95 | 21.643 | 8.09 |
| HPMC E4M | 0.17 | 1.887 | 0.71 |
| PEG 300 | 0.55 | 6.104 | 2.28 |
| Hydroxypropyl | 0.4 | 4.440 | 1.66 |
| Cellulose | | | |
| (NISSO HPC SSL) | | | |
| Sucralose | 0.24 | 2.664 | 1.00 |
| Magnasweet MM100 | 0.24 | 2.664 | 1.00 |
| HPMC E50 | 0.24 | 2.664 | 1.00 |
| PEO WSR N80 NF | 3.06 | 33.962 | 12.69 |
| total dry | 9.01 | 100.000 | N/A |
| total wet | 24.11 | N/A | 100.00 |
| FILM TARGET | 235.04 | N/A | N/A |
| WEIGHT (60 mg API) | | | |

### Example 17

In the following example, film matrix of lot 164-Ap made of poorly soluble API where a portion of Apomorphine is solubilized in acid condition, the rest of the API is suspended. The PEG 300 was used as solubilizer, permeation co-enhancer and as plasticizer for the film.

| | Theoretical | Composition | Composition |
|---|---|---|---|
| DESCRIPTION | quantity | % dry | % wet |
| lot# 164-Ap | (g) | (w/w) | (w/w) |
| PEG 300 | 0.54 | 7.42 | 2.42 |
| Purified Water | 15 | N/A | 67.33 |
| Succinic acid | 0.8 | 10.99 | 3.59 |
| Labrafil M2125CS | 0.117 | 1.61 | 0.53 |
| Tween 20 | 0.513 | 7.05 | 2.30 |
| Apomorphine | 1.8 | 24.74 | 8.08 |
| HPMC E4M | 0.171 | 2.35 | 0.77 |
| Hydroxypropyl Cellulose | 0.172 | 2.36 | 0.77 |
| (NISSO HPC SSL)) | | | |
| Sucralose | 0.232 | 3.19 | 1.04 |
| Ammonium Glycyrrhizinate | 0.232 | 3.19 | 1.04 |
| (Magnasweet MM100) | | | |
| Hypromellose 2910 USP | 0.2 | 2.75 | 0.90 |
| (HPMC E50) | | | |
| Polyethylene Oxide (Polyox | 2 | 27.48 | 8.98 |
| Sentry WSR N80 NF) | | | |
| Hydrolyzed med viscosity | 0.5 | 6.87 | 2.24 |
| guar gum | | | |
| Total | 22.277 | 100.00 | 100.00 |
| | Target weight | 242.57 | |
| | for (60 mg | | |
| | API) | | |

### Example 18

In the following example, film matrix of lot 164-Bu made of poorly soluble API where a portion of Buprenorphine is solubilized in acid condition, the rest is suspended. The propylene glycol was used as solubilizer and permeation co-enhancer and as plasticizer for the film.

| | Theoretical | Composition | Composition |
|---|---|---|---|
| DESCRIPTION | quantity | % dry | % wet |
| lot# 164-Bu | (g) | (w/w) | (w/w) |
| Propylene glycol | 0.54 | 7.42 | 2.42 |
| Purified Water | 15 | N/A | 67.33 |
| Citric acid | 0.8 | 10.99 | 3.59 |
| Labrafil M2125CS | 0.117 | 1.61 | 0.53 |
| Tween 20 | 0.513 | 7.05 | 2.30 |
| Buprenorphine | 1.8 | 24.74 | 8.08 |
| HPMC E4M | 0.171 | 2.35 | 0.77 |
| Hydroxypropyl Cellulose | 0.172 | 2.36 | 0.77 |
| (NISSO HPC SSL)) | | | |
| Sucralose | 0.232 | 3.19 | 1.04 |
| Ammonium Glycyrrhizinate | 0.232 | 3.19 | 1.04 |
| (Magnasweet MM100) | | | |
| Hypromellose 2910 USP | 0.2 | 2.75 | 0.90 |
| (HPMC E50) | | | |
| Polyethylene Oxide (Polyox | 2.5 | 34.35 | 11.22 |
| Sentry WSR N80 NF) | | | |
| Total | 22.277 | 100.00 | 100.00 |
| | Target weight | 242.57 | |
| | for (60 mg | | |
| | API) | | |

In the following example, film matrix of lot 164-Vi made of water soluble API where Ascorbic acid is solubilized in neutral condition. The glycerol was used as permeation co-enhancer and as plasticizer for the film.

| | Theoretical | Composition | Composition |
|---|---|---|---|
| DESCRIPTION | quantity | % dry | % wet |
| lot# 164-Vi | (g) | (w/w) | (w/w) |
| Glycerol | 0.54 | 7.92 | 2.48 |
| Purified Water | 15 | N/A | 68.76 |
| Labrafil M2125CS | 0.117 | 1.72 | 0.54 |
| Tween 20 | 0.25 | 3.67 | 1.15 |
| Ascorbic acid | 2.4 | 35.22 | 11.00 |
| HPMC E4M | 0.171 | 2.51 | 0.78 |
| Hydroxypropyl | 0.172 | 2.52 | 0.79 |
| Cellulose | | | |
| (NISSO HPC SSL)) | | | |
| Sucralose | 0.232 | 3.40 | 1.06 |
| Ammonium | 0.232 | 3.40 | 1.06 |
| Glycyrrhizinate | | | |
| (Magnasweet MM100) | | | |
| Hypromellose | 0.2 | 2.94 | 0.92 |
| 2910 USP (HPMC E50) | | | |
| Polyethylene Oxide | 2.5 | 36.69 | 11.46 |
| (Polyox Sentry | | | |
| WSR N80 NF) | | | |
| Total | 21.81 | 100 | 100 |
| Target weight | 255.53 | | |
| for (90 mg API) | | | |

### Example 19

Film matrix of lot 164-44 made in neutral condition where the API is maintained fully suspended with the assistance of surfactant and viscosity increasing agent to provide an increased surface area of API but less than the solubilized prototype.

| DESCRIPTION lot# 164-44 | wet % | Qt (g) | Dry % | mg/film |
|---|---|---|---|---|
| Glycerin (Glycerol USP) | 1.27 | 3.81 | 4.28 | 9.40 |
| Purified water USP | 70.3 | 210.9 | N/A | N/A |
| Hydroxylated Soy Lecithin | 2.7 | 8.1 | 9.09 | 19.98 |
| (SOLEC ™ S) | | | | |
| Maropitant Free Base | 8.11 | 24.33 | 27.31 | 60 |
| Hydroxypropyl | 0.81 | 2.43 | 2.73 | 5.99 |
| Methylcellulose (Metolose | | | | |
| 90SH-4000SR) | | | | |
| Hydroxypropyl Cellulose | 2.7 | 8.1 | 9.09 | 19.98 |
| (NISSO HPC SSL) | | | | |
| Sucralose | 1.09 | 3.27 | 3.67 | 8.06 |
| Ammonium Glycyrrhizinate | 1.09 | 3.27 | 3.67 | 8.06 |
| (Magnasweet MM100) | | | | |
| Yeast Extract (Ajinomoto | 0.27 | 0.81 | 0.91 | 2.00 |
| Brand Savorboost ™ BK) | | | | |
| Natural Liver Powdered | 1.6 | 4.8 | 5.39 | 11.84 |
| Flavor (PF.Inc PC-0050) | | | | |
| Hypromellose 2910 USP | 0.86 | 2.58 | 2.90 | 6.36 |
| (HPMC E50) | | | | |
| Polyethylene Oxide (Polyox | 9.2 | 27.6 | 30.98 | 68.06 |
| Sentry WSR N80 NF) | | | | |
| TOTAL WET | 100.00 | 300 | N/A | N/A |
| TOTAL DRY | N/A | 89.1 | 100.00 | 219.73 |

### Example 20

In this example, a solubilized pharmaceutical active phase is prepared by solubilizing the API (DMT) combined and mixed in solubilizing system made up of at least one of the following: methanol or ethanol.

Next, the soluble excipients are added and mixed followed by the addition of a viscosity increasing agent/suspending agent/polymer matrix to create the blend.

Deaeration of the blend and coating follows at a certain gap on the support liner, and at the end the coated material is placed in the oven for drying. Due to the size of the film multiple coating layer might be required to ensure good evaporation of the liquid.

| | | | |
|---|---|---|---|
| | Excipient | % wet | % dry |
| Ingredients | Function | (w/w) | (w/w) |

Lot#: 172-019

| | | | |
|---|---|---|---|
| water | Solvent | 26.609 | — |
| methanol | Solubilizer | 26.609 | — |
| DMT | API | 19.013 | 40.642 |
| citric acid | Stabilizer | 2.668 | 5.703 |
| FD&C Yellow No. 06 powder | Dye | 0.006 | 0.013 |
| Plasdone S-630 | Film former polymer | 1.382 | 2.954 |
| Nisso HPC SSL | Suspending agent | 3.017 | 6.449 |
| | Film former polymer | | |
| PVP K90 | Film former polymer | 20.696 | 44.239 |

Lot#: 169-094

| | | | |
|---|---|---|---|
| water | Solvent | 17.663 | — |
| methanol | Solubilizer | 41.213 | — |
| DMT | API | 14.723 | 35.802 |
| citric acid | Stabilizer | 2.066 | 5.024 |
| Neotame | Sweetener | 0.098 | 0.239 |
| Sucralose | Sweetener | 0.615 | 1.495 |
| Bitterness Suppresser | Taste masking agent | 1.276 | 3.102 |
| Strawberry flavor | flavor | 0.654 | 1.591 |
| FD&C Yellow No. 06 powder | Dye | 0.016 | 0.040 |
| PEO WRS80 | Film former polymer | 17.377 | 42.255 |
| HPMC E50 | Suspending agent | 1.963 | 4.772 |
| | Film former polymer | | |

Lot#: 169-076

| | Excipient | % wet | % dry |
|---|---|---|---|
| Ingredients | Function | (w/w) | (w/w) |
| water | Solvent | 18.120 | — |
| methanol | Solubilizer | 42.279 | — |
| DMT | API | 12.835 | 32.410 |
| citric acid | Stabilizer | 1.797 | 4.537 |
| Magnasweet | Sweetener | 0.101 | 0.254 |
| Acesulfame potassium | Sweetener | 0.631 | 1.593 |
| Bitterness Suppresser | Taste masking agent | 1.309 | 3.305 |
| Vanilla flavor | flavor | 0.671 | 1.695 |
| FD&C Yellow No. 10 powder | Dye | 0.017 | 0.042 |
| Plasdone S-63 | Film former polymer | 6.567 | 16.583 |
| Nisso HPC-L | Suspending agent | 6.567 | 16.583 |
| | Film former polymer | | |
| HPMC E50 | Film former polymer | 2.013 | 5.084 |
| D-Maltitol | Pore former | 4.698 | 11.862 |

### Manufacturing Procedure:

The oral film is produced by dissolving all the soluble ingredients and or dispersible ingredients in the media of choice and mixing until all excipients are solubilized and or dispersed yielding a uniform blend. The active ingredient is then added to the blend and solubilized or dispersed, depending on the formulation. The blend is mixed until get uniform and then polymers are added to the blend and mixed until completely dissolved. Once dissolved and homogenous, the mixing speed is lowered to allow degassing of the blend.

Preparation of a film product typically involves casting or otherwise thinly spreading the liquid film formulation on a substrate, drying (e.g., evaporating) all or most of the solvent(s) from the film to produce a thin, semi-solid/solid film sheet of material, and cutting the film sheet into individual unit dosage forms for packaging or processing.

The above description is considered that of the preferred embodiment(s) only. Modifications of these embodiments will occur by those skilled in the art and by those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. An oral film formulation comprising:
i. suspended Maropitant or a salt thereof;
ii. an aqueous phase consisting of water and at least one surfactant;
iii. at least one viscosity increasing agent/suspending agent,
iv. at least one solubilizer.

2. The oral film formulation of claim 1, wherein the Maropitant or a salt thereof is solubilized in a solubilizing system which includes at least one solubilizer.

3. The oral film formulation of claim 2, wherein the ratio of Maropitant or a salt thereof to solubilizer is from 10:3 to 10:60.

4. The oral film formulation of claim 1, wherein the Maropitant or a salt thereof is solubilized up to 5%.

5. The oral film formulation of claim 1, wherein the Maropitant or a salt thereof is solubilized up to 40%.

6. The oral film formulation of claim 1, wherein the solubilizer is a benzene derivative selected from the group consisting of benzyl alcohol, benzyl acetate, benzaldehyde, and other benzene derivatives.

7. The oral film formulation of claim 1, wherein the solubilizer is a fatty acid selected from the group consisting of alpha-Linolenic acid, alpha-Linoleic acid, arachidonic acid, palmitoleic acid, gadoleic acid, Caproic acid, heptanoic acid, 11-Dodecenoic acid, Caprylic acid, Ricinoleic acid and caproleic acid, other fatty acids, and other fatty acids derivatives.

8. The oral film formulation of claim 1, wherein the solubilizer is selected from the group consisting of a non-volatile excipient, a volatile excipient, and a combination of volatile and non-volatile excipients.

9. The oral film formulation of claim 1, wherein the solubilizer is a surfactant.

10. The oral film formulation of claim 1, wherein the solubilizer is a solubility enhancer.

11. The oral film formulation of claim 1, wherein the solubilizer encompasses oils, including the possibility of essential oils.

12. The oral film formulation of claim 1, wherein the solubilizer is a pH modulator.

13. The oral film formulation of claim 12, wherein the pH modulator can be either an acid excipient, basic excipient or a buffer excipient.

14. The oral film formulation of claim 1, having a film surface pH of not more than 9, and preferably not more than 8.5.

15. The oral film formulation of claim 1, having a film blend mix pH of not more than 10, and preferably not more than 8.5.

16. A method for preparing an oral film comprising the steps of:
a. Formulating a film-forming composition, said composition comprising:
i. Maropitant or a salt thereof
ii. An aqueous phase consisting of water and at least one surfactant
iii. at least one viscosity increasing agent/suspending agent, and
b. Solubilizing a portion of Maropitant or a salt thereof in a solubilizing system made up of at least one solubilizer;
c. Preparing the aqueous phase with at least one surfactant;
d. Combine the phases;
e. Adding a second amount of Maropitant of a salt thereof;
d. Adding a viscosity increasing agent/suspending agent to stabilize the emulsion;
e. optionally adding at least one low viscosity solubilizing polymer;
f. Optionally, adding one or more acceptable excipients selected from the group consisting of co-surfactants, permeation co-enhancers, flavoring agents, flavor enhancers, sweeteners, colorants, plasticizers, stabilizers, antifoaming agents, antioxidants, preservatives, and other excipients;
g. Adding film forming polymers;
h. Casting onto a substrate to form a film;
i. Drying the film to remove the solvent; and
j. Cutting the dried film into individual oral film units.

17. An oral film comprising solubilized API in solubilizer, suspended API, a surfactant system and a viscosity increasing agent/suspending agent, wherein the portion of suspended API exceeds the portion of the API in solubilizer, and wherein the oral film dosage forms delivers up to 80 mg of API or 120 mg of API salt.

18. The oral film of claim 17, further comprising one or more acceptable excipients selected from the group consisting of co-surfactants, permeation co-enhancers, flavoring agents, flavor enhancers, sweeteners, colorants, plasticizers, stabilizers, antioxidants, antifoaming agents preservatives, and other excipients.

19. The oral film of claim 17, wherein the solubilizer is selected from the group consisting of a non-volatile excipient, a volatile excipient, and a combination of volatile and non-volatile excipients.

20. The oral film of any one of the previous claims 1-19, wherein the oral film is suitable for use in animals.
